(19) United States
(12) Patent Application Publication
(10) Pub. No.: US 2023/0332197 A1
(43) Pub. Date: Oct. 19, 2023

(54) NUCLEOSIDES AND NUCLEOTIDES WITH 3' VINYL BLOCKING GROUP

(71) Applicant: Illumina Singapore Pte. Ltd., Singapore (SG)

(72) Inventors: Ramesh Neelakandan, Singapore (SG); Daniel Hartoyo Lukamto, Singapore (SG); Jean-Alexandre Richard, Singapore (SG); Yin Nah Teo, Singapore (SG); Xiangyuan Yang, Singapore (SG); Sadeer Salam, Singapore (SG); Jing Liang, Singapore (SG); Saurabh Nirantar, Singapore (SG)

(21) Appl. No.: 18/192,291

(22) Filed: Mar. 29, 2023

Related U.S. Application Data
(60) Provisional application No. 63/325,880, filed on Mar. 31, 2022.

(51) Int. Cl. C12P 19/34; C07H 19/01

(52) U.S. Cl. CPC C12P 19/34; C07H 19/01

(57) ABSTRACT

Embodiments of the present disclosure relate to nucleotide and nucleoside molecules with an optionally substituted 3′vinyl blocking group. Also provided herein are methods to prepare such nucleotide and nucleoside molecules. Additionally, the present disclosure provides methods of using such blocked nucleosides and nucleotides in oligonucleotide synthesis and sequencing.

### CROSS-REFERENCE TO RELATED APPLICATIONS

This application claims the benefit of priority to U.S. Provisional Application No. 63/325,880, filed Mar. 31, 2022, the content of which is incorporated by reference in its entirety.

### FIELD

The present disclosure generally relates to nucleotides, nucleosides, or oligonucleotides comprising an optionally substituted 3′vinyl blocking groups. Methods of preparing the 3′ vinyl blocked nucleotides, nucleosides, or oligonucleotides are also disclosed.

### REFERENCE TO SEQUENCE LISTING

The present application is being filed along with a Sequence Listing in electronic format. The Sequencing Listing is provided as a file entitled Sequence_listing_ILLINC_725A.xml, created Mar. 17, 2023, which is 3.2 kB in size. The information in the electronic format of the Sequence Listing in incorporated herein by reference in its entirety.

### BACKGROUND

Advances in the study of molecules have been led, in part, by improvement in technologies used to characterize the molecules or their biological reactions. In particular, the study of the nucleic acids DNA and RNA has benefited from developing technologies used for sequence analysis and the study of hybridization events.

An example of the technologies that have improved the study of nucleic acids is the development of fabricated arrays of immobilized nucleic acids. These arrays consist typically of a high-density matrix of polynucleotides immobilized onto a solid support material. See, e.g., Jacobs et al., Combinatorial chemistry-applications of light-directed chemical synthesis, Trends Biotech. 12: 19-26 (1994), which describes ways of assembling the nucleic acids using a chemically sensitized glass surface protected by a mask, but exposed at defined areas to allow attachment of suitably modified nucleotide phosphoramidites. Fabricated arrays can also be manufactured by the technique of “spotting” known polynucleotides onto a solid support at predetermined positions (e.g., Stimpson et al., real-time detection of DNA hybridization and melting on oligonucleotide arrays by using optical wave guides, Proc. Natl. Acad. Sci. 92: 6379-83 (1995)).

One way of determining the nucleotide sequence of a nucleic acid bound to an array is called “sequencing by synthesis” or “SBS”. This technique for determining the sequence of DNA ideally requires the controlled (i.e., one at a time) incorporation of the correct complementary nucleotide opposite the nucleic acid being sequenced. This allows for accurate sequencing by adding nucleotides in multiple cycles as each nucleotide residue is sequenced one at a time, thus preventing an uncontrolled series of incorporations from occurring. The incorporated nucleotide is read using an appropriate label attached thereto before removal of the label moiety and the subsequent next round of sequencing.

In order to ensure that only a single incorporation occurs, a structural modification (“protecting group” or “blocking group”) is included in each labeled nucleotide that is added to the growing chain to ensure that only one nucleotide is incorporated. After the nucleotide with the protecting group has been added, the protecting group is then removed, under reaction conditions which do not interfere with the integrity of the DNA being sequenced. The sequencing cycle can then continue with the incorporation of the next protected, labeled nucleotide.

There are many limitations on the types of 3′ blocking groups that can be added onto a nucleotide and still be suitable. The protecting group should prevent additional nucleotide molecules from being added to the polynucleotide chain whilst simultaneously being easily removable from the sugar moiety without causing damage to the polynucleotide chain. Furthermore, the modified nucleotide needs to be compatible with the polymerase or another appropriate enzyme used to incorporate it into the polynucleotide chain. The ideal protecting group must therefore exhibit long-term stability, be efficiently incorporated by the polymerase enzyme, cause blocking of secondary or further nucleotide incorporation, and be removable under mild conditions that do not cause damage to the polynucleotide structure, preferably under aqueous conditions.

Reversible protecting groups have been described previously. For example, Metzker et al., (Nucleic Acids Research, 22 (20): 4259-4267, 1994) discloses the synthesis and use of eight 3′-modified 2-deoxyribonucleoside 5′-triphosphates (3′-modified dNTPs) and testing in two DNA template assays for incorporation activity. WO 2002/029003 describes a sequencing method which may include the use of an allyl protecting group to cap the 3′-OH group on a growing strand of DNA in a polymerase reaction. In addition, the development of a number of reversible protecting groups and methods of deprotecting them under DNA compatible conditions was previously reported in International Application Publication Nos. WO 2004/018497 and WO 2014/139596, each of which is hereby incorporated by reference in its entirety.

### SUMMARY

One aspect of the present disclosure relates to a nucleoside or nucleotide having the structure of Formula (I):

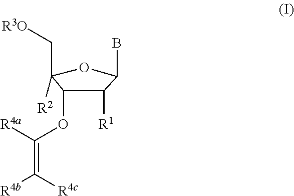

or a salt thereof, wherein
B comprises a nucleobase;
R¹ is H, hydroxy, —OR⁵, halo, or a hydroxy protecting group;
R² is H;
R³ is H, a hydroxy protecting group, or —OR³ is a monophosphate, diphosphate, triphosphate or phosphorothioate;
each of R^{4a}, R^{4b} and R^{4c}, is independently H, halo, C₁-C₃ alkyl, C₁-C₃ haloalkyl, C₁-C₃ hydroxyalkyl, azido, optionally substituted phenyl, optionally substituted 4 to 6 membered heteroaryl, optionally substituted C₃-C₇ cycloalkyl, or optionally substituted 3 to 7 membered heterocyclyl; and
R⁵ is C₁-C₆ alkyl or C₁-C₆ haloalkyl, or when R¹ is —OR⁵, R⁵ and R² together with the atoms to which they are attached form a four to seven membered heterocycle containing one oxygen atom.

Another aspect of the present disclosure relates to a method of controlled synthesis of an oligonucleotide or polynucleotide, comprising:
contacting a nucleotide of Formula (I) as described herein with the oligonucleotide or polynucleotide in the presence of a polymerase; and
incorporating the nucleotide to the 3′ end of the oligonucleotide or polynucleotide.

A further aspect of the present disclosure relates to a method of synthesizing a nucleotide of Formula (Id):

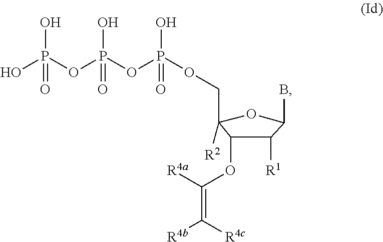

or a salt thereof, comprising:
reacting a nucleoside of Formula (Ie):

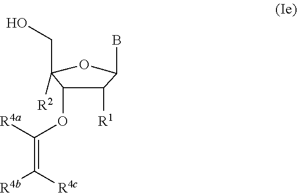

with

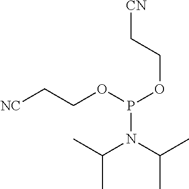 [SMILES: CC(C)N(C(C)C)P(OCCC#N)OCCC#N]

in the presence of a peroxide to form a first intermediate of Formula (Ie-1):

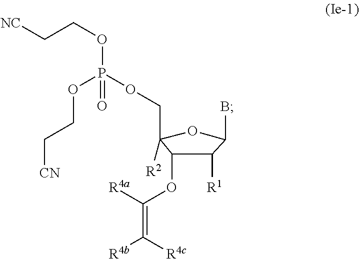

reacting the first intermediate with 1,8-diazabicyclo[5.4.0]undec-7-ene (DBU) to form a second intermediate of Formula (Ie-2):

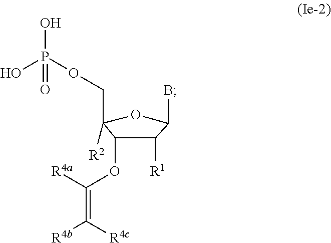

reacting the second intermediate with 2-methyl-1H-imidazole to form the third intermediate of Formula (Ie-3):

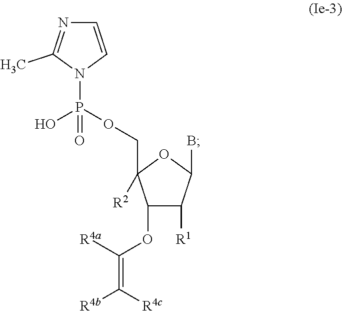

and
reacting the third intermediate with pyrophosphate H₂P₂O₇ to generate the compound of Formula (Id);
wherein B comprises a nucleobase;
R¹ is H, hydroxy, —OR⁵, halo, or a hydroxy protecting group;
R² is H;
each of R^{4a}, R^{4b} and R^{4c}, is independently H, halo, C₁-C₃ alkyl, C₁-C₃ haloalkyl, C₁-C₃ hydroxyalkyl, azido, optionally substituted phenyl, optionally substituted 4 to 6 membered heteroaryl, optionally substituted C₃-C₇ cycloalkyl, or optionally substituted 3 to 7 membered heterocyclyl; and
R⁵ is C₁-C₆ alkyl or C₁-C₆ haloalkyl, or alternatively, when R¹ is —OR⁵, R⁵ and R² together with the atoms to which they are attached form a four to seven membered heterocycle containing one oxygen atom.

### BRIEF DESCRIPTION OF THE DRAWINGS

FIG. 1 illustrates a three-dimensional visualization of a nucleotide interacting with an oligonucleotide in the presence of a DNA polymerase.

FIG. 2 illustrates relative sizes of azidomethyl (—CH₂N₃) and vinyl (—CH═CH₂) blocking groups.

FIG. 3 is a gel electrophoresis image demonstrating the capability of various DNA polymerases to incorporate a 3′ vinyl deoxythymidine triphosphate (dTTP) into an oligonucleotide.

FIG. 4 illustrates a plot of UV absorbance over time of 3′ vinyl thymidine monophosphate (TMP) and 3′OH TMP after exposure to a tetrazine reagent.

FIG. 5 shows a gel electrophoresis image demonstrating terminal deoxynucleotidyl transferase (TdT)'s capability to incorporate a single 3′ vinyl dTTP.

### DETAILED DESCRIPTION

Embodiments of the present disclosure relate to nucleosides and nucleotides with 3′ vinyl group. This blocking group has demonstrated fast deblocking rate and ease of incorporation into oligonucleotide via biosynthesis.

### Definitions

Unless defined otherwise, all technical and scientific terms used herein have the same meaning as is commonly understood by one of ordinary skill in the art. The use of the term “including” as well as other forms, such as “include,” “includes,” and “included,” is not limiting. The use of the term “having” as well as other forms, such as “have,” “has,” and “had,” is not limiting. As used in this specification, whether in a transitional phrase or in the body of the claim, the terms “comprise(s)” and “comprising” are to be interpreted as having an open-ended meaning. That is, the above terms are to be interpreted synonymously with the phrases “having at least” or “including at least.” For example, when used in the context of a process, the term “comprising” means that the process includes at least the recited steps, but may include additional steps. When used in the context of a compound, composition, or device, the term “comprising” means that the compound, composition, or device includes at least the recited features or components, but may also include additional features or components.

As used herein, common organic abbreviations are defined as follows:
° C. Temperature in degrees Centigrade
AZM Azidomethyl
dATP Deoxyadenosine triphosphate
dCTP Deoxycytidine triphosphate
dGTP Deoxyguanosine triphosphate
dTTP Deoxythymidine triphosphate
ddNTP Dideoxynucleotide triphosphate
ffN Fully functionalized nucleotide
RT Room temperature
SBS Sequencing by Synthesis
TMP Thymidine monophosphate

As used herein, the term “covalently attached” or “covalently bonded” refers to the forming of a chemical bonding that is characterized by the sharing of pairs of electrons between atoms. For example, a covalently attached polymer coating refers to a polymer coating that forms chemical bonds with a functionalized surface of a substrate, as compared to attachment to the surface via other means, for example, adhesion or electrostatic interaction. It will be appreciated that polymers that are attached covalently to a surface can also be bonded via means in addition to covalent attachment.

As used herein, any “R” group(s) represent substituents that can be attached to the indicated atom. An R group may be substituted or unsubstituted. If two “R” groups are described as “together with the atoms to which they are attached” forming a ring or ring system, it means that the collective unit of the atoms, intervening bonds and the two R groups are the recited ring. For example, when the following substructure is present:

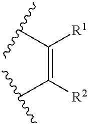

and R¹ and R² are defined as selected from the group consisting of hydrogen and alkyl, or R¹ and R² together with the atoms to which they are attached form an aryl or carbocyclyl, it is meant that R¹ and R² can be selected from hydrogen or alkyl, or alternatively, the substructure has structure:

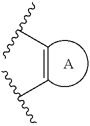

where A is an aryl ring or a carbocyclyl containing the depicted double bond.

It is to be understood that certain radical naming conventions can include either a mono-radical or a di-radical, depending on the context. For example, where a substituent requires two points of attachment to the rest of the molecule, it is understood that the substituent is a di-radical. For example, a substituent identified as alkyl that requires two points of attachment includes di-radicals such as —CH₂—, —CH₂CH₂—, —CH₂CH(CH₃)CH₂—, and the like. Other radical naming conventions clearly indicate that the radical is a di-radical such as “alkylene” or “alkenylene.”

The term “halogen” or “halo,” as used herein, means any one of the radio-stable atoms of column 7 of the Periodic Table of the Elements, e.g., fluorine, chlorine, bromine, or iodine, with fluorine and chlorine being preferred.

As used herein, “Cₐ to C_{b}” in which “a” and “b” are integers refer to the number of carbon atoms in an alkyl, alkenyl or alkynyl group, or the number of ring atoms of a cycloalkyl or aryl group. That is, the alkyl, the alkenyl, the alkynyl, the ring of the cycloalkyl, and ring of the aryl can contain from “a” to “b”, inclusive, carbon atoms. For example, a “C₁ to C₄ alkyl” group refers to all alkyl groups having from 1 to 4 carbons, that is, CH₃—, CH₃CH₂—, CH₃CH₂CH₂—, (CH₃)₂CH—, CH₃CH₂CH₂CH₂—, CH₃CH₂CH(CH₃)— and (CH₃)₃C—; a C₃ to C₄ cycloalkyl group refers to all cycloalkyl groups having from 3 to 4 carbon atoms, that is, cyclopropyl and cyclobutyl. Similarly, a “4 to 6 membered heterocyclyl” group refers to all heterocyclyl groups with 4 to 6 total ring atoms, for example, azetidine, oxetane, oxazoline, pyrrolidine, piperidine, piperazine, morpholine, and the like. If no “a” and “b” are designated with regard to an alkyl, alkenyl, alkynyl, cycloalkyl, or aryl group, the broadest range described in these definitions is to be assumed. As used herein, the term “C₁-C₆” includes C₁, C₂, C₃, C₄, C₅ and C₆, and a range defined by any of the two numbers. For example, C₁-C₆ alkyl includes C₁, C₂, C₃, C₄, C₅ and C₆ alkyl, C₂-C₆ alkyl, C₁-C₃ alkyl, etc. Similarly, C₂-C₆ alkenyl includes C₂, C₃, C₄, C₅ and C₆ alkenyl, C₂-C₈ alkenyl, C₃-C₄ alkenyl, etc.; and C₂-C₆ alkynyl includes C₂, C₃, C₄, C₅ and C₆ alkynyl, C₂-C₈ alkynyl, C₃-C₄ alkynyl, etc. C₃-C₈ cycloalkyl each includes hydrocarbon ring containing 3, 4, 5, 6, 7 and 8 carbon atoms, or a range defined by any of the two numbers, such as C₃-C₇ cycloalkyl or C₅-C₆ cycloalkyl.

As used herein, “alkyl” refers to a straight or branched hydrocarbon chain that is fully saturated (i.e., contains no double or triple bonds). The alkyl group may have 1 to 20 carbon atoms (whenever it appears herein, a numerical range such as “1 to 20” refers to each integer in the given range; e.g., “1 to 20 carbon atoms” means that the alkyl group may consist of 1 carbon atom, 2 carbon atoms, 3 carbon atoms, etc., up to and including 20 carbon atoms, although the present definition also covers the occurrence of the term “alkyl” where no numerical range is designated). The alkyl group may also be a medium size alkyl having 1 to 9 carbon atoms. The alkyl group could also be a lower alkyl having 1 to 6 carbon atoms. The alkyl group may be designated as “C₁-C₄ alkyl” or similar designations. By way of example only, “C₁-C₆ alkyl” indicates that there are one to six carbon atoms in the alkyl chain, i.e., the alkyl chain is selected from the group consisting of methyl, ethyl, propyl, iso-propyl, n-butyl, iso-butyl, sec-butyl, and t-butyl. Typical alkyl groups include, but are in no way limited to, methyl, ethyl, propyl, isopropyl, butyl, isobutyl, tertiary butyl, pentyl, hexyl, and the like.

As used herein, “alkoxy” refers to the formula —OR wherein R is an alkyl as is defined above, such as “C₁-C₉ alkoxy”, including but not limited to methoxy, ethoxy, n-propoxy, 1-methylethoxy (isopropoxy), n-butoxy, iso-butoxy, sec-butoxy, and tert-butoxy, and the like.

As used herein, “alkenyl” refers to a straight or branched hydrocarbon chain containing one or more double bonds. The alkenyl group may have 2 to 20 carbon atoms, although the present definition also covers the occurrence of the term “alkenyl” where no numerical range is designated. The alkenyl group may also be a medium size alkenyl having 2 to 9 carbon atoms. The alkenyl group could also be a lower alkenyl having 2 to 6 carbon atoms. The alkenyl group may be designated as “C₂-C₆ alkenyl” or similar designations. By way of example only, “C₂-C₆ alkenyl” indicates that there are two to six carbon atoms in the alkenyl chain, i.e., the alkenyl chain is selected from the group consisting of ethenyl, propen-1-yl, propen-2-yl, propen-3-yl, buten-1-yl, buten-2-yl, buten-3-yl, buten-4-yl, 1-methyl-propen-1-yl, 2-methyl-propen-1-yl, 1-ethyl-ethen-1-yl, 2-methyl-propen-3-yl, buta-1,3-dienyl, buta-1,2,-dienyl, and buta-1,2-dien-4-yl. Typical alkenyl groups include, but are in no way limited to, ethenyl, propenyl, butenyl, pentenyl, and hexenyl, and the like.

As used herein, “alkynyl” refers to a straight or branched hydrocarbon chain containing one or more triple bonds. The alkynyl group may have 2 to 20 carbon atoms, although the present definition also covers the occurrence of the term “alkynyl” where no numerical range is designated. The alkynyl group may also be a medium size alkynyl having 2 to 9 carbon atoms. The alkynyl group could also be a lower alkynyl having 2 to 6 carbon atoms. The alkynyl group may be designated as “C₂-C₆ alkynyl” or similar designations. By way of example only, “C₂-C₆ alkynyl” indicates that there are two to six carbon atoms in the alkynyl chain, i.e., the alkynyl chain is selected from the group consisting of ethynyl, propyn-1-yl, propyn-2-yl, butyn-1-yl, butyn-3-yl, butyn-4-yl, and 2-butynyl. Typical alkynyl groups include, but are in no way limited to, ethynyl, propynyl, butynyl, pentynyl, and hexynyl, and the like.

As used herein, “heteroalkyl” refers to a straight or branched hydrocarbon chain containing one or more heteroatoms, that is, an element other than carbon, including but not limited to, nitrogen, oxygen and sulfur, in the chain backbone. The heteroalkyl group may have 1 to 20 carbon atoms, although the present definition also covers the occurrence of the term “heteroalkyl” where no numerical range is designated. The heteroalkyl group may also be a medium size heteroalkyl having 1 to 9 carbon atoms. The heteroalkyl group could also be a lower heteroalkyl having 1 to 6 carbon atoms. The heteroalkyl group may be designated as “C₁-C₆ heteroalkyl” or similar designations. The heteroalkyl group may contain one or more heteroatoms. By way of example only, “C₄-C₆ heteroalkyl” indicates that there are four to six carbon atoms in the heteroalkyl chain and additionally one or more heteroatoms in the backbone of the chain.

The term “aromatic” refers to a ring or ring system having a conjugated pi electron system and includes both carbocyclic aromatic (e.g., phenyl) and heterocyclic aromatic groups (e.g., pyridine). The term includes monocyclic or fused-ring polycyclic (i.e., rings which share adjacent pairs of atoms) groups provided that the entire ring system is aromatic.

As used herein, “aryl” refers to an aromatic ring or ring system (i.e., two or more fused rings that share two adjacent carbon atoms) containing only carbon in the ring backbone. When the aryl is a ring system, every ring in the system is aromatic. The aryl group may have 6 to 18 carbon atoms, although the present definition also covers the occurrence of the term “aryl” where no numerical range is designated. In some embodiments, the aryl group has 6 to 10 carbon atoms. The aryl group may be designated as “C₆-C₁₀ aryl,” “C₆ or C₁₀ aryl,” or similar designations. Examples of aryl groups include, but are not limited to, phenyl, naphthyl, azulenyl, and anthracenyl.

An “aralkyl” or “arylalkyl” is an aryl group connected, as a substituent, via an alkylene group, such as “C₇₋₁₄ aralkyl” and the like, including but not limited to benzyl, 2-phenylethyl, 3-phenylpropyl, and naphthylalkyl. In some cases, the alkylene group is a lower alkylene group (i.e., a C₁-C₆ alkylene group).

As used herein, “heteroaryl” refers to an aromatic ring or ring system (i.e., two or more fused rings that share two adjacent atoms) that contain(s) one or more heteroatoms, that is, an element other than carbon, including but not limited to, nitrogen, oxygen and sulfur, in the ring backbone. When the heteroaryl is a ring system, every ring in the system is aromatic. The heteroaryl group may have 5-18 ring members (i.e., the number of atoms making up the ring backbone, including carbon atoms and heteroatoms), although the present definition also covers the occurrence of the term “heteroaryl” where no numerical range is designated. In some embodiments, the heteroaryl group has 5 to 10 ring members or 5 to 7 ring members. The heteroaryl group may be designated as “5-7 membered heteroaryl,” “5-10 membered heteroaryl,” or similar designations. Examples of heteroaryl rings include, but are not limited to, furyl, thienyl, phthalazinyl, pyrrolyl, oxazolyl, thiazolyl, imidazolyl, pyrazolyl, isoxazolyl, isothiazolyl, triazolyl, thiadiazolyl, pyridinyl, pyridazinyl, pyrimidinyl, pyrazinyl, triazinyl, quinolinyl, isoquinlinyl, benzimidazolyl, benzoxazolyl, benzothiazolyl, indolyl, isoindolyl, and benzothienyl.

A “heteroaralkyl” or “heteroarylalkyl” is heteroaryl group connected, as a substituent, via an alkylene group. Examples include but are not limited to 2-thienylmethyl, 3-thienylmethyl, furylmethyl, thienylethyl, pyrrolylalkyl, pyridylalkyl, isoxazollylalkyl, and imidazolylalkyl. In some cases, the alkylene group is a lower alkylene group (i.e., a C₁-C₆ alkylene group).

As used herein, “carbocyclyl” means a non-aromatic cyclic ring or ring system containing only carbon atoms in the ring system backbone. When the carbocyclyl is a ring system, two or more rings may be joined together in a fused, bridged or spiro-connected fashion. Carbocyclyls may have any degree of saturation provided that at least one ring in a ring system is not aromatic. Thus, carbocyclyls include cycloalkyls, cycloalkenyls, and cycloalkynyls. The carbocyclyl group may have 3 to 20 carbon atoms, although the present definition also covers the occurrence of the term “carbocyclyl” where no numerical range is designated. The carbocyclyl group may also be a medium size carbocyclyl having 3 to 10 carbon atoms. The carbocyclyl group could also be a carbocyclyl having 3 to 6 carbon atoms. The carbocyclyl group may be designated as “C₃-C₆ carbocyclyl” or similar designations. Examples of carbocyclyl rings include, but are not limited to, cyclopropyl, cyclobutyl, cyclopentyl, cyclohexyl, cyclohexenyl, 2,3-dihydro-indene, bicycle[2.2.2]octanyl, adamantyl, and spiro[4.4]nonanyl.

As used herein, “cycloalkyl” means a fully saturated carbocyclyl ring or ring system. Examples include cyclopropyl, cyclobutyl, cyclopentyl, and cyclohexyl.

As used herein, “heterocyclyl” means a non-aromatic cyclic ring or ring system containing at least one heteroatom in the ring backbone. Heterocyclyls may be joined together in a fused, bridged or spiro-connected fashion. Heterocyclyls may have any degree of saturation provided that at least one ring in the ring system is not aromatic. The heteroatom(s) may be present in either a non-aromatic or aromatic ring in the ring system. The heterocyclyl group may have 3 to 20 ring members (i.e., the number of atoms making up the ring backbone, including carbon atoms and heteroatoms), although the present definition also covers the occurrence of the term “heterocyclyl” where no numerical range is designated. The heterocyclyl group may also be a medium size heterocyclyl having 3 to 10 ring members. The heterocyclyl group could also be a heterocyclyl having 3 to 6 ring members. The heterocyclyl group may be designated as “3-6 membered heterocyclyl” or similar designations. In preferred six membered monocyclic heterocyclyls, the heteroatom(s) are selected from one up to three of O, N or S, and in preferred five membered monocyclic heterocyclyls, the heteroatom(s) are selected from one or two heteroatoms selected from O, N, or S. Examples of heterocyclyl rings include, but are not limited to, azepinyl, acridinyl, carbazolyl, cinnolinyl, dioxolanyl, imidazolinyl, imidazolidinyl, morpholinyl, oxiranyl, oxepanyl, thiepanyl, piperidinyl, piperazinyl, dioxopiperazinyl, pyrrolidinyl, pyrrolidonyl, pyrrolidionyl, 4-piperidonyl, pyrazolinyl, pyrazolidinyl, 1,3-dioxinyl, 1,3-dioxanyl, 1,4-dioxinyl, 1,4-dioxanyl, 1,3-oxathianyl, 1,4-oxathiinyl, 1,4-oxathianyl, 2H-1,2-oxazinyl, trioxanyl, hexahydro-1,3,5-triazinyl, 1,3-dioxolyl, 1,3-dioxolanyl, 1,3-dithiolyl, 1,3-dithiolanyl, isoxazolinyl, isoxazolidinyl, oxazolinyl, oxazolidinyl, oxazolidinonyl, thiazolinyl, thiazolidinyl, 1,3-oxathiolanyl, indolinyl, isoindolinyl, tetrahydrofuranyl, tetrahydropyranyl, tetrahydrothiophenyl, tetrahydrothiopyranyl, tetrahydro-1,4-thiazinyl, thiamorpholinyl, dihydrobenzofuranyl, benzimidazolidinyl, and tetrahydroquinoline.

An “O-carboxy” group refers to a “—OC(═O)R” group in which R is selected from hydrogen, C₁-C₆ alkyl, C₂-C₆ alkenyl, C₂-C₆ alkynyl, C₃-C₇ carbocyclyl, C₆-C₁₀ aryl, 5-10 membered heteroaryl, and 3-10 membered heterocyclyl, as defined herein.

A “C-carboxy” group refers to a “—C(═O)OR” group in which R is selected from the group consisting of hydrogen, C₁-C₆ alkyl, C₂-C₆ alkenyl, C₂-C₆ alkynyl, C₃-C₇ carbocyclyl, C₆-C₁₀ aryl, 5-10 membered heteroaryl, and 3-10 membered heterocyclyl, as defined herein. A non-limiting example includes carboxyl (i.e., —C(═O)OH).

A “sulfonyl” group refers to an “—SO₂R” group in which R is selected from hydrogen, C₁-C₆ alkyl, C₂-C₆ alkenyl, C₂-C₆ alkynyl, C₃-C₇ carbocyclyl, C₆-C₁₀ aryl, 5-10 membered heteroaryl, and 3-10 membered heterocyclyl, as defined herein.

A “sulfino” group refers to a “—S(═O)OH” group.

A “S-sulfonamido” group refers to a “—SO₂NR_{A}R_{B}” group in which R_{A} and R_{B} are each independently selected from hydrogen, C₁-C₆ alkyl, C₂-C₆ alkenyl, C₂-C₆ alkynyl, C₃-C₇ carbocyclyl, C₆-C₁₀ aryl, 5-10 membered heteroaryl, and 3-10 membered heterocyclyl, as defined herein.

An “N-sulfonamido” group refers to a “—N(R_{A})SO₂R_{B}” group in which R_{A} and R_{b} are each independently selected from hydrogen, C₁-C₆ alkyl, C₂-C₆ alkenyl, C₂-C₆ alkynyl, C₃-C₇ carbocyclyl, C₆-C₁₀ aryl, 5-10 membered heteroaryl, and 3-10 membered heterocyclyl, as defined herein.

A “C-amido” group refers to a “—C(═O)NR_{A}R_{B}” group in which R_{A} and R_{B} are each independently selected from hydrogen, C₁-C₆ alkyl, C₂-C₆ alkenyl, C₂-C₆ alkynyl, C₃-C₇ carbocyclyl, C₆-C₁₀ aryl, 5-10 membered heteroaryl, and 3-10 membered heterocyclyl, as defined herein.

An “N-amido” group refers to a “—N(R_{A})C(═O)R_{B}” group in which R_{A} and R_{B} are each independently selected from hydrogen, C₁-C₆ alkyl, C₂-C₆ alkenyl, C₂-C₆ alkynyl, C₃-C₇ carbocyclyl, C₆-C₁₀ aryl, 5-10 membered heteroaryl, and 3-10 membered heterocyclyl, as defined herein.

An “amino” group refers to a “—NR_{A}R_{B}” group in which R_{A} and R_{B} are each independently selected from hydrogen, C₁-C₆ alkyl, C₂-C₆ alkenyl, C₂-C₆ alkynyl, C₃-C₇ carbocyclyl, C₆-C₁₀ aryl, 5-10 membered heteroaryl, and 3-10 membered heterocyclyl, as defined herein. A non-limiting example includes free amino (i.e., —NH₂).

An “aminoalkyl” group refers to an amino group connected via an alkylene group.

An “alkoxyalkyl” group refers to an alkoxy group connected via an alkylene group, such as a C₂-C₈ alkoxyalkyl or (C₁-C₆ alkoxy)C₁-C₆ alkyl, for example, —(CH₂)₁₋₃—OCH₃.

As used herein, a substituted group is derived from the unsubstituted parent group in which there has been an exchange of one or more hydrogen atoms for another atom or group. Unless otherwise indicated, when a group is deemed to be “substituted,” it is meant that the group is substituted with one or more substituents independently selected from C₁-C₆ alkyl, C₁-C₆ alkenyl, C₁-C₆ alkynyl, C₁-C₆ heteroalkyl, C₃-C₇ carbocyclyl (optionally substituted with halo, C₁-C₆ alkyl, C₁-C₆ alkoxy, C₁-C₆ haloalkyl, and C₁-C₆ haloalkoxy), C₃-C₇-carbocyclyl-C₁-C₆-alkyl (optionally substituted with halo, C₁-C₆ alkyl, C₁-C₆ alkoxy, C₁-C₆ haloalkyl, and C₁-C₆ haloalkoxy), 3-10 membered heterocyclyl (optionally substituted with halo, C₁-C₆ alkyl, C₁-C₆ alkoxy, C₁-C₆ haloalkyl, and C₁-C₆ haloalkoxy), 3-10 membered heterocyclyl-C₁-C₆-alkyl (optionally substituted with halo, C₁-C₆ alkyl, C₁-C₆ alkoxy, C₁-C₆ haloalkyl, and C₁-C₆ haloalkoxy), aryl (optionally substituted with halo, C₁-C₆ alkyl, C₁-C₆ alkoxy, C₁-C₆ haloalkyl, and C₁-C₆ haloalkoxy), aryl(C₁-C₆)alkyl (optionally substituted with halo, C₁-C₆ alkyl, C₁-C₆ alkoxy, C₁-C₆ haloalkyl, and C₁-C₆ haloalkoxy), 5-10 membered heteroaryl (optionally substituted with halo, C₁-C₆ alkyl, C₁-C₆ alkoxy, C₁-C₆ haloalkyl, and C₁-C₆ haloalkoxy), 5-10 membered heteroaryl(C₁-C₆)alkyl (optionally substituted with halo, C₁-C₆ alkyl, C₁-C₆ alkoxy, C₁-C₆ haloalkyl, and C₁-C₆ haloalkoxy), halo, —CN, hydroxy, C₁-C₆ alkoxy, C₁-C₆ alkoxy(C₁-C₆)alkyl (i.e., ether), aryloxy, sulfhydryl (mercapto), halo(C₁-C₆)alkyl (e.g., —CF₃), halo(C₁-C₆)alkoxy (e.g., —OCF₃), C₁-C₆ alkylthio, arylthio, amino, amino(C₁-C₆)alkyl, nitro, 0-carbamyl, N-carbamyl, 0-thiocarbamyl, N-thiocarbamyl, C-amido, N-amido, S-sulfonamido, N-sulfonamido, C-carboxy, O-carboxy, acyl, cyanato, isocyanato, thiocyanato, isothiocyanato, sulfinyl, sulfonyl, —SO₃H, sulfino, —OSO₂C₁₋₄alkyl, and oxo (═O). Wherever a group is described as “optionally substituted” that group can be substituted with the above substituents.

As used herein, the term “hydroxy” refers to a —OH group.

As used herein, the term “cyano” group refers to a “—CN” group.

As used herein, the term “azido” refers to a —N₃ group.

As used herein, the term “azidomethyl” refers to a “—CH₂N₃” group. In the context of a 3′ blocking group, the term “3′ azidomethyl” means that the azidomethyl group that is covalently attached to the 3′ oxygen of the ribose or deoxyribose ring of the nucleoside or nucleotide.

As used herein the term “vinyl” refers to a “—CH═CH₂”. In the context of a 3′ blocking group, the term “3′ vinyl” means that the vinyl group that is covalently attached to the 3′ oxygen of the ribose or deoxyribose ring of the nucleoside or nucleotide.

As used herein, a “nucleotide” includes a nitrogen containing heterocyclic base, a sugar, and one or more phosphate groups. They are monomeric units of a nucleic acid sequence. In RNA, the sugar is a ribose, and in DNA a deoxyribose, i.e. a sugar lacking a hydroxyl group that is present in ribose. The nitrogen containing heterocyclic base can be purine or pyrimidine base. Purine bases include adenine (A) and guanine (G), and modified derivatives or analogs thereof, such as deazapurine or 7-deazapurine (e.g., deaza adenine, 7-deaza adenine, deaza guanine, or 7-deaza guanine). Pyrimidine bases include cytosine (C), thymine (T), and uracil (U), and modified derivatives or analogs thereof. The C-1 atom of deoxyribose is bonded to N-1 of a pyrimidine or N-9 of a purine.

As used herein, a “nucleoside” is structurally similar to a nucleotide but is missing the phosphate moieties. An example of a nucleoside analogue would be one in which the label is linked to the base and there is no phosphate group attached to the sugar molecule. The term “nucleoside” is used herein in its ordinary sense as understood by those skilled in the art. Examples include, but are not limited to, a ribonucleoside comprising a ribose moiety and a deoxyribonucleoside comprising a deoxyribose moiety. A modified pentose moiety is a pentose moiety in which an oxygen atom has been replaced with a carbon and/or a carbon has been replaced with a sulfur or an oxygen atom. A “nucleoside” is a monomer that can have a substituted base and/or sugar moiety. Additionally, a nucleoside can be incorporated into larger DNA and/or RNA polymers and oligomers.

The term “purine base” is used herein in its ordinary sense as understood by those skilled in the art and includes its tautomers. Similarly, the term “pyrimidine base” is used herein in its ordinary sense as understood by those skilled in the art and includes its tautomers. A non-limiting list of optionally substituted purine-bases includes purine, deazapurine, adenine, deaza adenine, guanine, deaza guanine, 7-deaza adenine, 7-deaza guanine, hypoxanthine, xanthine, alloxanthine, 7-alkylguanine (e.g., 7-methylguanine), theobromine, caffeine, uric acid and isoguanine. Examples of pyrimidine bases include, but are not limited to, cytosine, thymine, uracil, 5,6-dihydrouracil and 5-alkylcytosine (e.g., 5-methylcytosine).

As used herein, when an oligonucleotide or polynucleotide is described as “comprising” a nucleoside or nucleotide described herein, it means that the nucleoside or nucleotide described herein forms a covalent bond with the oligonucleotide or polynucleotide. Similarly, when a nucleoside or nucleotide is described as part of an oligonucleotide or polynucleotide, such as “incorporated into” an oligonucleotide or polynucleotide, it means that the nucleoside or nucleotide described herein forms a covalent bond with the oligonucleotide or polynucleotide. In some such embodiments, the covalent bond is formed between a 3′ hydroxy group of the oligonucleotide or polynucleotide with the 5′ phosphate group of a nucleotide described herein as a phosphodiester bond between the 3′ carbon atom of the oligonucleotide or polynucleotide and the 5′ carbon atom of the nucleotide.

As used herein, “derivative” or “analogue” means a synthetic nucleotide or nucleoside derivative having modified base moieties and/or modified sugar moieties. Such derivatives and analogs are discussed in, e.g., Scheit, Nucleotide Analogs (John Wiley & Son, 1980) and Uhlman et al., Antisense oligonucleotides: a new therapeutic principle, Chemical Reviews 90:543-84 (1990). Nucleotide analogs can also comprise modified phosphodiester linkages, including phosphorothioate, phosphorodithioate, alkyl-phosphonate, phosphoranilidate and phosphoramidate linkages. “Derivative”, “analog” and “modified” as used herein, may be used interchangeably, and are encompassed by the terms “nucleotide” and “nucleoside” defined herein.

As used herein, the term “phosphate” is used in its ordinary sense as understood by those skilled in the art, and includes its protonated forms (for example,

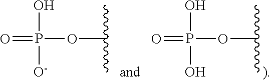 [SMILES: *OP(=O)([O-])O ; *OP(=O)(O)O]

As used herein, the terms “monophosphate,” “diphosphate,” and “triphosphate” are used in their ordinary sense as understood by those skilled in the art and include protonated forms.

The terms “protecting group” and “protecting groups” as used herein refer to any atom or group of atoms that is added to a molecule in order to prevent existing groups in the molecule from undergoing unwanted chemical reactions. Sometimes, “protecting group” and “blocking group” can be used interchangeably. Examples of protecting group moieties are described in T. W. Greene and P. G. M. Wuts, Protective Groups in Organic Synthesis, 4. Ed. John Wiley & Sons, 2006, and in J. F. W. McOmie, Protective Groups in Organic Chemistry Plenum Press, 1973, both of which are hereby incorporated by reference for the limited purpose of disclosing suitable protecting groups. The protecting group moiety may be chosen in such a way, that they are stable to certain reaction conditions and readily removed at a convenient stage using methodology known from the art. A non-limiting list of protecting groups include benzyl (Bn); substituted benzyl; alkylcarbonyls (e.g., t-butoxycarbonyl (BOC), acetyl (i.e., —C(═O)CH₃ or Ac), or isobutyryl (iBu); arylalkylcarbonyls (e.g., benzyloxycarbonyl or benzoyl (i.e., —C(═O)Ph or Bz)); substituted methyl ether (e.g., methoxymethyl ether (MOM)); substituted ethyl ether (e.g., methoxyethyl ether (MOE); a substituted benzyl ether; tetrahydropyranyl ether; silyl ethers (e.g., trimethylsilyl (TMS), triethylsilyl, triisopropylsilyl, t-butyldimethylsilyl (TBDMS), tri-iso-propylsilyloxymethyl (TOM), or t-butyldiphenylsilyl); esters (e.g., benzoate ester); carbonates (e.g., methoxymethylcarbonate); sulfonates (e.g., tosylate or mesylate); acyclic ketal (e.g., dimethyl acetal); cyclic ketals (e.g., 1,3-dioxane or 1,3-dioxolanes); acyclic acetal; cyclic acetal; acyclic hemiacetal; cyclic hemiacetal; cyclic dithioketals (e.g., 1,3-dithiane or 1,3-dithiolane); and triarylmethyl groups (e.g., trityl; monomethoxytrityl (MMTr); 4,4′-dimethoxytrityl (DMTr); or 4,4′,4″-trimethoxytrityl (TMTr)).

Examples of hydroxy protecting groups include without limitation, acetyl, t-butyl, t-butoxymethyl, methoxymethyl, tetrahydropyranyl, 1-ethoxyethyl, 1-(2-chloroethoxy)ethyl, p-chlorophenyl, 2,4-dinitrophenyl, benzyl, 2,6-dichlorobenzyl, diphenylmethyl, p-nitrobenzyl, bis(2-acetoxyethoxy)methyl (ACE), 2-trimethylsilylethyl, trimethylsilyl, triethylsilyl, t-butyldimethylsilyl, t-butyldiphenylsilyl, triphenylsilyl, [(triisopropylsilyl)oxy]methyl (TOM), benzoylformate, chloroacetyl, trichloroacetyl, trifluoro-acetyl, pivaloyl, benzoyl, p-phenylbenzoyl, 9-fluorenylmethyl carbonate, mesylate, tosylate, triphenylmethyl (trityl), monomethoxytrityl, dimethoxytrityl (DMT), trimethoxytrityl, 1(2-fluorophenyl)-4-methoxypiperidin-4-yl (FPMP), 9-phenylxanthine-9-yl (Pixyl) and 9-(p-methoxyphenyl)xanthine-9-yl (MOX). Wherein more commonly used hydroxyl protecting groups include without limitation, benzyl, 2,6-dichlorobenzyl, t-butyldimethylsilyl, t-butyldiphenylsilyl, benzoyl, mesylate, tosylate, dimethoxytrityl (DMT), 9-phenylxanthine-9-yl (Pixyl) and 9-(p-methoxyphenyl)xanthine-9-yl (MOX).

### 3′ Vinyl Blocking Groups

Oligonucleotide synthesis encompasses both chemical and biochemical methods of manufacturing an oligonucleotide. Illumina's suite of products includes both short and long oligo/polymers of DNA/RNA, which may be synthesized chemically using nucleoside phosphoramidites on solid supports, and/or biochemically using polymerases/ligases and nucleoside triphosphates. To achieve these methods of oligonucleotide synthesis, a protecting group strategy may be helpful to ensure clean, efficient and robust productions.

Disclosed herein are novel protecting group strategies for use in oligonucleotide synthesis. The synthetic or semi-synthetic manipulation of nucleosides in oligonucleotide synthesis may involve a protecting group strategy which includes a series of incorporation stages followed by deprotection stages. One way to achieve this is to place stable and tolerant blocking group on the 3′ hydroxy of the nucleosides/nucleotides. Furthermore, it may be helpful if the blocking groups themselves are tolerant to the oligonucleotide synthesis conditions. It may additionally be helpful if the deprotection stages and subsequent nucleotide steps are efficient and reliable.

Certain blocking groups have been previously described. For example, azidomethyl (AZM) has been used for biochemical synthesis with polymerases, and acid-labile groups such as 4,4′-dimethoxytrityl (DMT) has been used for solid-phase chemical synthesis. However, these groups may be too unstable for long-term storage and/or require the use of acid in production, which introduces side-reactions such as depurination, and may be detrimental to the synthesis of long oligonucleotides and genes. Furthermore, a more tolerant protecting group may allow orthogonal functional group tolerability.

Thus, a robust and tolerant protecting group with clean and reliable deprotection mechanism, while utilizing neutral pH conditions, may address some of the issues of previous blocking groups. One example strategy having fast and clean deprotection is the use of vinyl ethers as hydroxyl protecting groups. These groups are discussed in greater detail within Voronin et al., Examining the vinyl moiety as a protecting group for hydroxyl (—OH) functionality under basic conditions, Organic Chem. Frontiers, 2020, 7, 1334-42, incorporated herein by reference. Furthermore, vinyl group is also acid labile.

Alcohols may be protected as either ethers or esters, as discussed by P. G. W. Wuts et al., Greene's Protective Groups in Organic Synthesis, John Wiley & Sons, Inc., Hoboken, NJ, 4 edn., (2006), incorporated herein by reference. Of ethers and esters, ethers may be more tolerant to strong reactants including oxidizing agents and organometallic compounds. However, this property may also make ethers more difficult to deprotect. Furthermore, the use of ethers as protecting groups may involve expensive, harsh or hazardous reagents, strongly acidic conditions, long reaction times and tedious workup procedures. For instance, although alkyl and benzyl ethers are highly stable, their deprotections may involve hazardous reagents, such as BBr₃, BF₃·Et₂O, HgCl₂, 2,3-dichloro-5,6-dicyano-p-benzoquinone (DDQ), alkaline metals, or potentially explosive hydrogenation conditions. Acid-labile silyl ethers can be removed under mild conditions. However, the cost of silyl chlorides needed for the protection may be high and may introduce bulky substituents that interfere with polymerase activities in the biochemical synthesis of oligonucleotides. The simpler methoxymethyl (MOM) ethers may be used but the removal of MOM blocking group require rather harsh reaction conditions.

Alternatively, vinyl ethers may be a viable option to provide higher stability while allowing deprotection with safe and readily available tetrazine derivatives. The use of tetrazine derivatives is discussed further within B. L. Oliveira et al., Inverse electron demand Diels-Alder reactions in chemical biology, Bernardes. Chem. Soc. Rev. 2017, 46, 4895-950. The deprotection of vinyl ethers with tetrazine may involve relatively fast kinetics, approaching a ‘click’-type reaction, which has been termed inverse electron-demand Diels-Alder (IEDDA), under relatively low concentrations, for example within the μM range. The IEDDA is described in E. Jimenez-Moreno et al., Vinyl Ether/Tetrazine Pair for the Traceless Release of Alcohols in Cells, Angew. Chem. Int. Ed. 2017, 56, 243.

One aspect of the present disclosure relates to a nucleoside or nucleotide having the structure of Formula (I):

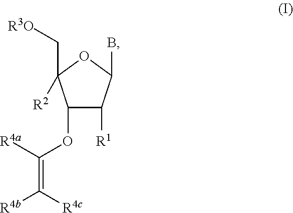

or a salt thereof, wherein
B comprises a nucleobase;
R¹ is H, hydroxy, —OR⁵, halo, or a hydroxy protecting group;
R² is H;
R³ is H, a hydroxy protecting group, or —OR³ is a monophosphate, diphosphate, triphosphate or phosphorothioate;
each of R^{4a}, R^{4b} and R^{4c} is independently H, halo, C₁-C₃ alkyl, C₁-C₃ haloalkyl, C₁-C₃ hydroxyalkyl, azido, optionally substituted phenyl, optionally substituted 4 to 6 membered heteroaryl, optionally substituted C₃-C₇ cycloalkyl, or optionally substituted 3 to 7 membered heterocyclyl; and
R⁵ is C₁-C₆ alkyl or C₁-C₆ haloalkyl, or alternative when R¹ is —OR⁵, R⁵ and R² together with the atoms to which they are attached form a four to seven membered heterocycle containing one oxygen atom.

In some embodiments of the nucleoside/nucleotide of Formula (I), each of R^{4a}, R^{4b} and R^{4c} is H, and the nucleoside or nucleotide has the structure of Formula (Ia):

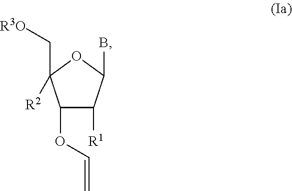

or a salt thereof. In other embodiments, at least one of R^{4a}, R^{4b} and R^{4c} is independently methyl, ethyl, n-propyl, isopropyl, fluoro, chloro, —CHF₂, —CH₂F, —CH₂C₁, —CHCl₂, or —CF₃. In some such embodiments, two of R^{4a}, R^{4b} and R^{4c} is H. In other embodiments, two of R^{4a}, R^{4b} and R^{4c} is independently methyl, ethyl, n-propyl, isopropyl, fluoro, chloro, —CHF₂, —CH₂F, —CH₂C₁, —CHCl₂, or —CF₃ and the remaining one of R^{4a}, R^{4b} and R^{4c} is H. In still other embodiments, each one of R^{4a}, R^{4b} and R^{4c} is independently methyl, ethyl, n-propyl, isopropyl, fluoro, chloro, —CHF₂, —CH₂F, —CH₂C₁, —CHCl₂, or —CF₃.

In some embodiments of the nucleoside/nucleotide of Formula (I), R¹ is H. In other embodiments, R¹ is hydroxy, or a hydroxy protecting group, for example, a hydroxy protecting group selected from the group consisting of

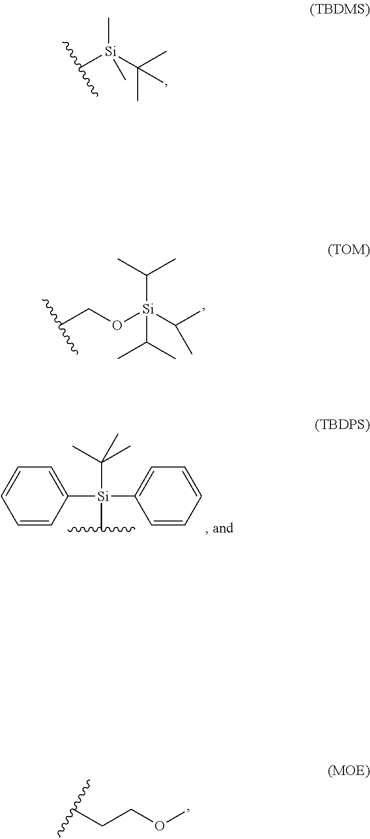

wherein the squiggly line shows the point of attachment of the protecting group to the 2′ oxygen atom. In another embodiment, R¹ is halo, such as fluoro. In still other embodiments, R¹ is —OR⁵. In some such embodiment, R⁵ is C₁-C₆ alkyl, (e.g., methyl, ethyl, n-propyl, isopropyl, n-butyl, or t-butyl). In one embodiment, R¹ is methoxy.

In some embodiments of the nucleoside/nucleotide of Formula (I), R² is H. In other embodiments, R¹ is —OR⁵ and R⁵ and R² together with the atoms to which they are attached form a four to seven membered heterocycle having the Formula (Ib):

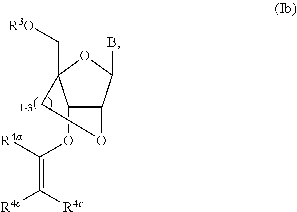

or a salt thereof. In further embodiment, R⁵ and R² together with the atoms to which they are attached form a four membered heterocycle having the Formula (Ib′):

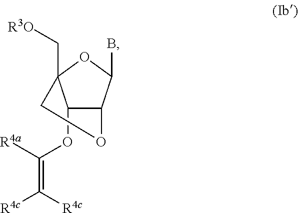

or a salt thereof. In further embodiments, each of R^{4a}, R^{4b} and R^{4c} is H, and the nucleoside or nucleotide has the structure of Formula (Ic):

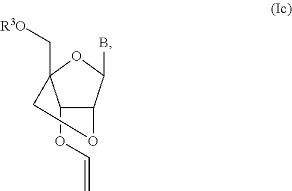

or a salt thereof.

In some embodiments of the nucleoside/nucleotide of Formula (I), (Ia), (Ib), (Ib′) or (Ic), —OR³ is a monophosphate, diphosphate or triphosphate. In further embodiments, the

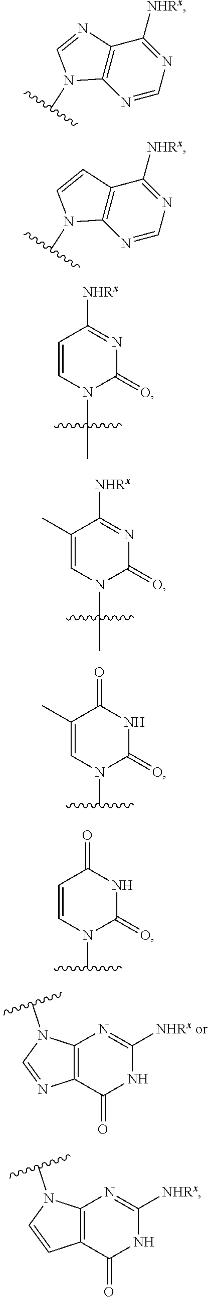

wherein R^{x} is H or an amino protecting group. In further embodiments, R^{x} is H, —C(═O)C₁₋₆ alkyl (e.g., Ac—C(═O)CH³, or iBu —C(═O)CH(CH₃)₂), or —C(═O)-phenyl (Bz). Alternatively, the hydrogen in —NHR^{x} is absent, and R^{x} is a divalent amino protecting group, such as ═CHN(CH₃)₂ (dmf). In further embodiments, B comprises

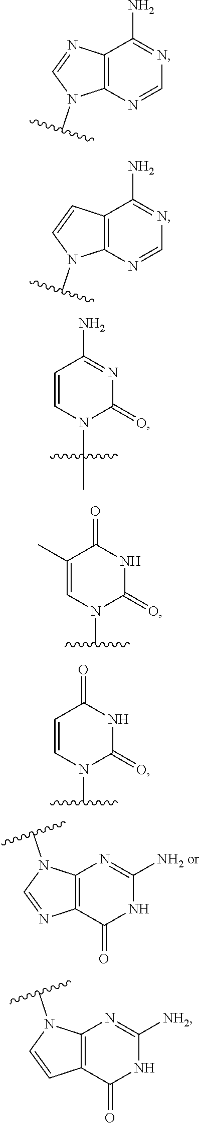

or optionally substituted derivatives and analogs thereof.

In further embodiments, B comprises a nucleobase covalently bounded to a detectable label, optionally through a linker, for example a cleavable linker. In some further embodiments, B is

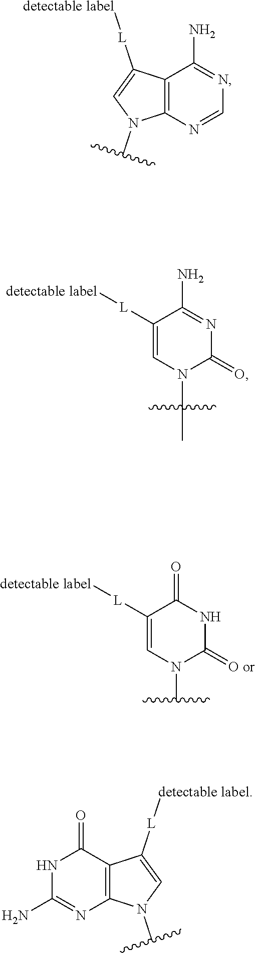

In any embodiments of the nucleoside or nucleotide described here, the nucleotide may be nucleotide triphosphate (i.e., —OR³ is a triphosphate).

### Linkers

The detectable label as disclosed herein may include a reactive linker group at one of the substituent positions for covalent attachment of the detectable label to the nucleoside/nucleotide described herein. Reactive linking groups are moieties capable of forming a bond (e.g., a covalent or non-covalent bond), in particular a covalent bond. In a particular embodiment the linker may be a cleavable linker. Use of the term “cleavable linker” is not meant to imply that the whole linker is required to be removed. The cleavage site can be located at a position on the linker that ensures that part of the linker remains attached to the dye and/or substrate moiety after cleavage. Cleavable linkers may be, by way of non-limiting example, electrophilically cleavable linkers, nucleophilically cleavable linkers, photocleavable linkers, cleavable under reductive conditions (for example disulfide or azide containing linkers), oxidative conditions, cleavable via use of safety-catch linkers and cleavable by elimination mechanisms. The use of a cleavable linker to attach the dye compound to a substrate moiety ensures that the label can, if required, be removed after detection, avoiding any interfering signal in downstream steps.

Useful linker groups may be found in PCT Publication No. WO2004/018493 (herein incorporated by reference), examples of which include linkers that may be cleaved using water-soluble phosphines or water-soluble transition metal catalysts formed from a transition metal and at least partially water-soluble ligands. In aqueous solution the latter form at least partially water-soluble transition metal complexes. Such cleavable linkers can be used to connect bases of nucleotides to labels such as the dyes set forth herein.

Particular linkers include those disclosed in PCT Publication No. WO2004/018493 (herein incorporated by reference) such as those that include moieties of the formulae:

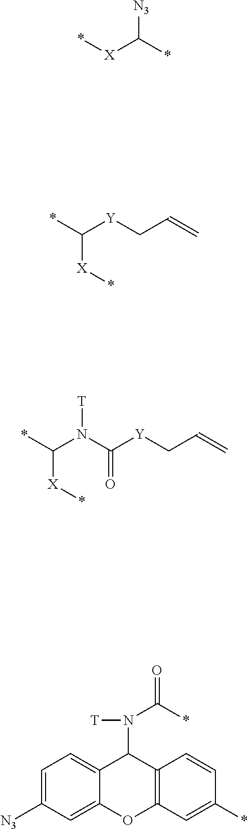

(wherein X is selected from the group comprising O, S, NH and NQ wherein Q is a C1-10 substituted or unsubstituted alkyl group, Y is selected from the group comprising O, S, NH and N(allyl), T is hydrogen or a C₁-C₁₀ substituted or unsubstituted alkyl group and * indicates where the moiety is connected to the remainder of the nucleotide or nucleoside). In some aspect, the linkers connect the bases of nucleotides to labels such as, for example, the dye compounds described herein.

Additional examples of linkers include those disclosed in U.S. Publication No. 2016/0040225 (herein incorporated by reference), such as those include moieties of the formulae:

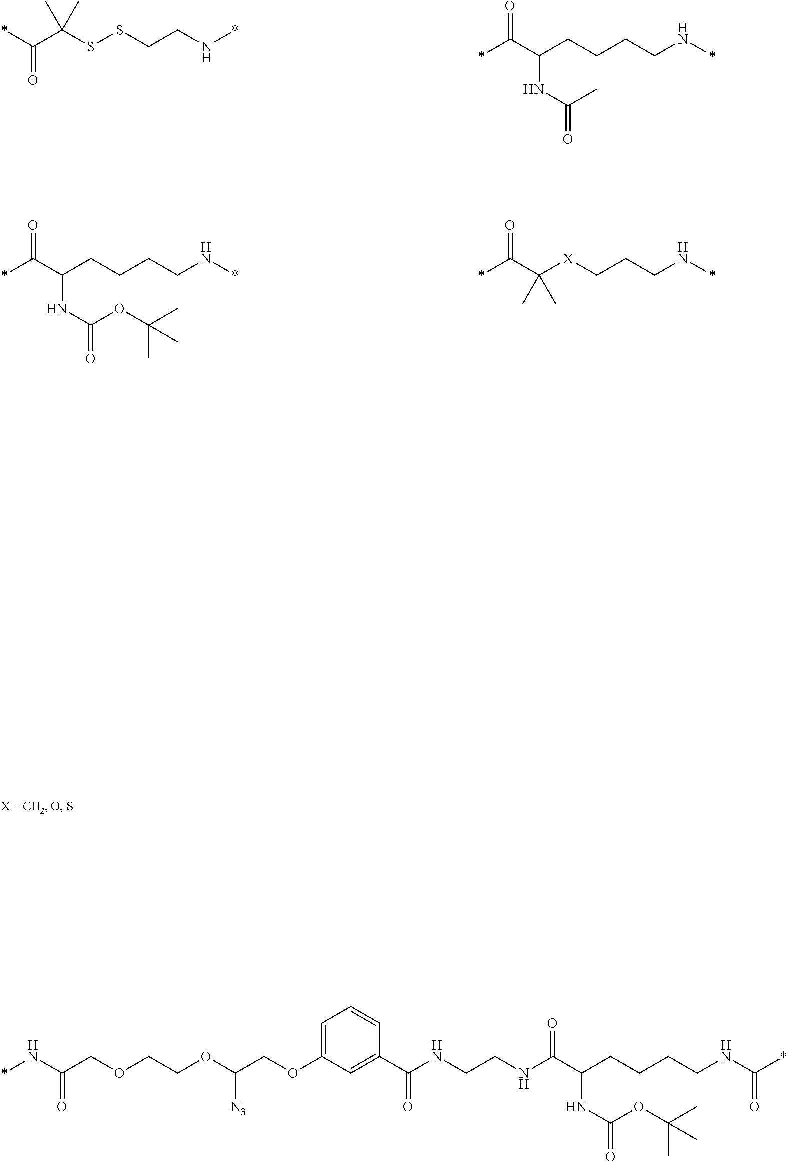

(wherein * indicates where the moiety is connected to the remainder of the nucleotide or nucleoside). The linker moieties illustrated herein may comprise the whole or partial linker structure between the nucleotides/nucleosides and the labels. The linker moieties illustrated herein may comprise the whole or partial linker structure between the nucleotides/nucleosides and the labels.

Additional examples of linkers include moieties of the formula:

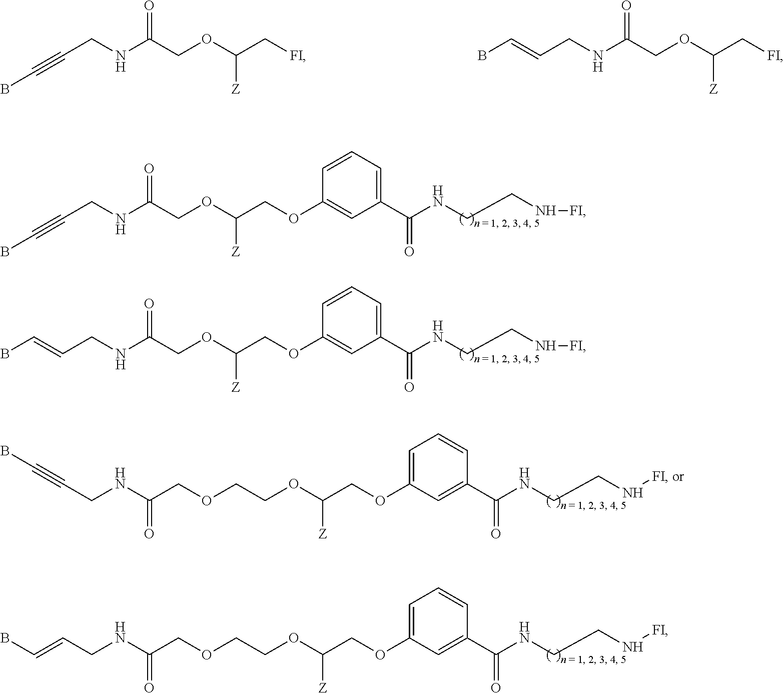

wherein B is a nucleobase; Z is —N₃ (azido), —O—C₁-C₆ alkyl, —O—C₂-C₆ alkenyl, or —O—C₂-C₆ alkynyl; and F1 comprises a dye moiety, which may contain additional linker structure. One of ordinary skill in the art understands that the dye compound described herein is covalently bounded to the linker by reacting a functional group of the dye compound (e.g., carboxyl) with a functional group of the linker (e.g., amino). In one embodiment, the cleavable linker comprises a vinyl group.

In particular embodiments, the length of the linker between the detectable label and the nucleobase can be altered, for example, by introducing a polyethylene glycol spacer group, thereby increasing the fluorescence intensity compared to the same fluorophore attached to the guanine base through other linkages known in the art. Exemplary linkers and their properties are set forth in PCT Publication No. WO2007020457 (herein incorporated by reference). When the detectable label is for use in any method of analysis which requires detection of a fluorescent dye label attached to a nucleoside/nucleotide, it is advantageous if the linker comprises a spacer group of formula —((CH₂)₂O)ₙ—, wherein n is an integer between 2 and 50, as described in WO 2007/020457.

A detectable label may be attached to any position on the nucleotide base, for example, through a linker. In particular embodiments, Watson-Crick base pairing can still be carried out for the resulting analog. Particular nucleobase labeling sites include the C₅ position of a pyrimidine base or the C₇ position of a 7-deaza purine base. As described above a linker group may be used to covalently attach a dye to the nucleoside or nucleotide.

The use of a blocking group allows polymerization to be controlled, such as by stopping extension when a labeled nucleotide is incorporated. If the blocking effect is reversible, for example, by way of non-limiting example by changing chemical conditions or by removal of a chemical block, extension can be stopped at certain points and then allowed to continue.

In a particular embodiment, the linker (between the detectable label and nucleotide) and blocking group are both present and are separate moieties. In particular embodiments, the linker and vinyl blocking group are both cleavable under the same or substantially similar conditions. Thus, deprotection and deblocking processes may be more efficient because only a single treatment will be required to remove both the detectable label and the blocking group. However, in some embodiments a linker and blocking group need not be cleavable under similar conditions, instead being individually cleavable under distinct conditions.

Non-limiting exemplary labeled nucleotides as described herein include:

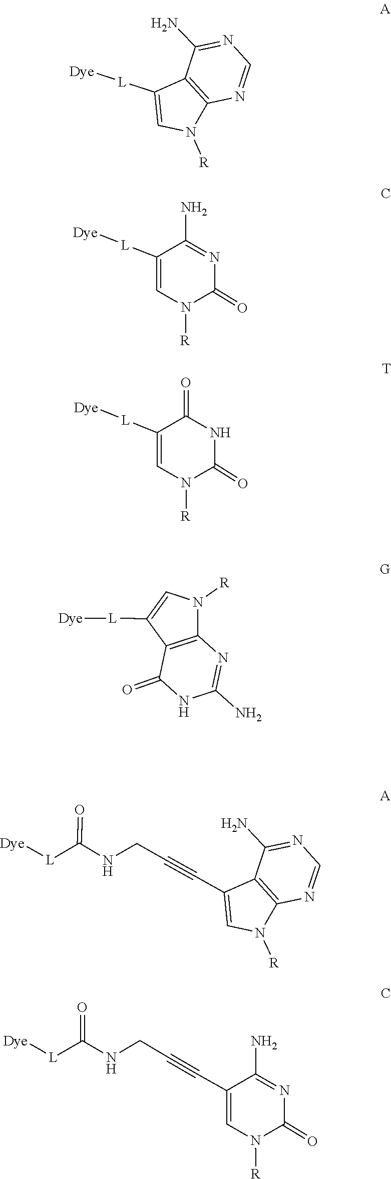

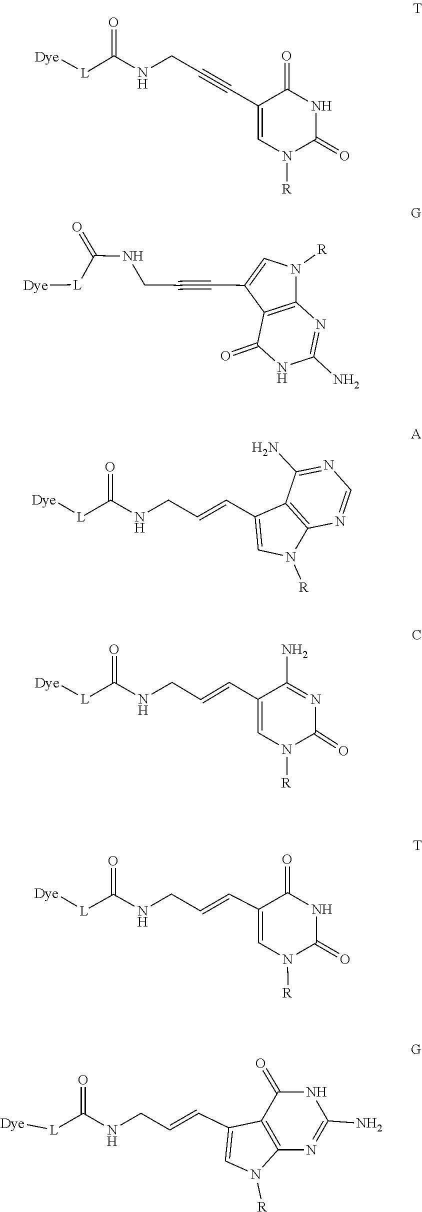

wherein L represents a linker and R represents a ribose or deoxyribose moiety with the 3′ vinyl blocking group as described herein, or a ribose or deoxyribose moiety with the 5′ position substituted with mono-, di- or tri- phosphates.

In some embodiments, non-limiting exemplary labeled nucleotides are shown below:

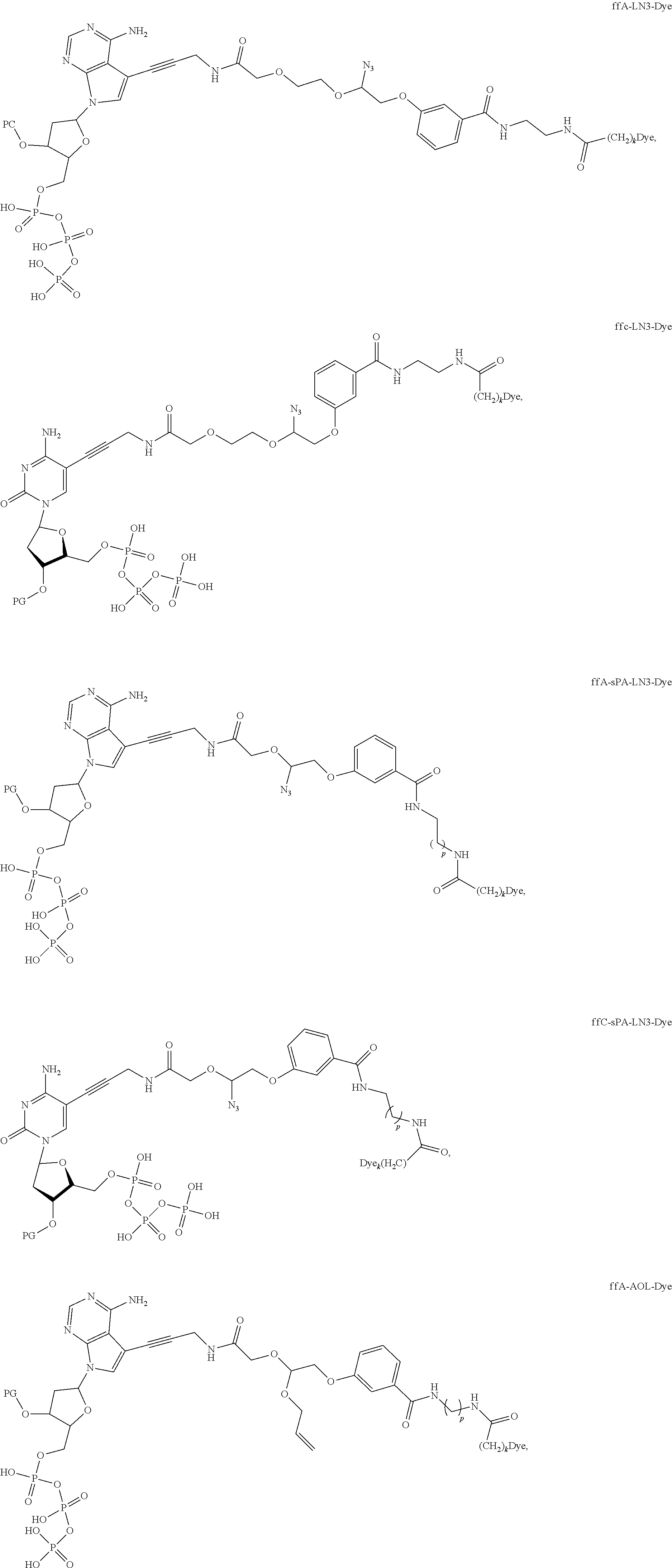

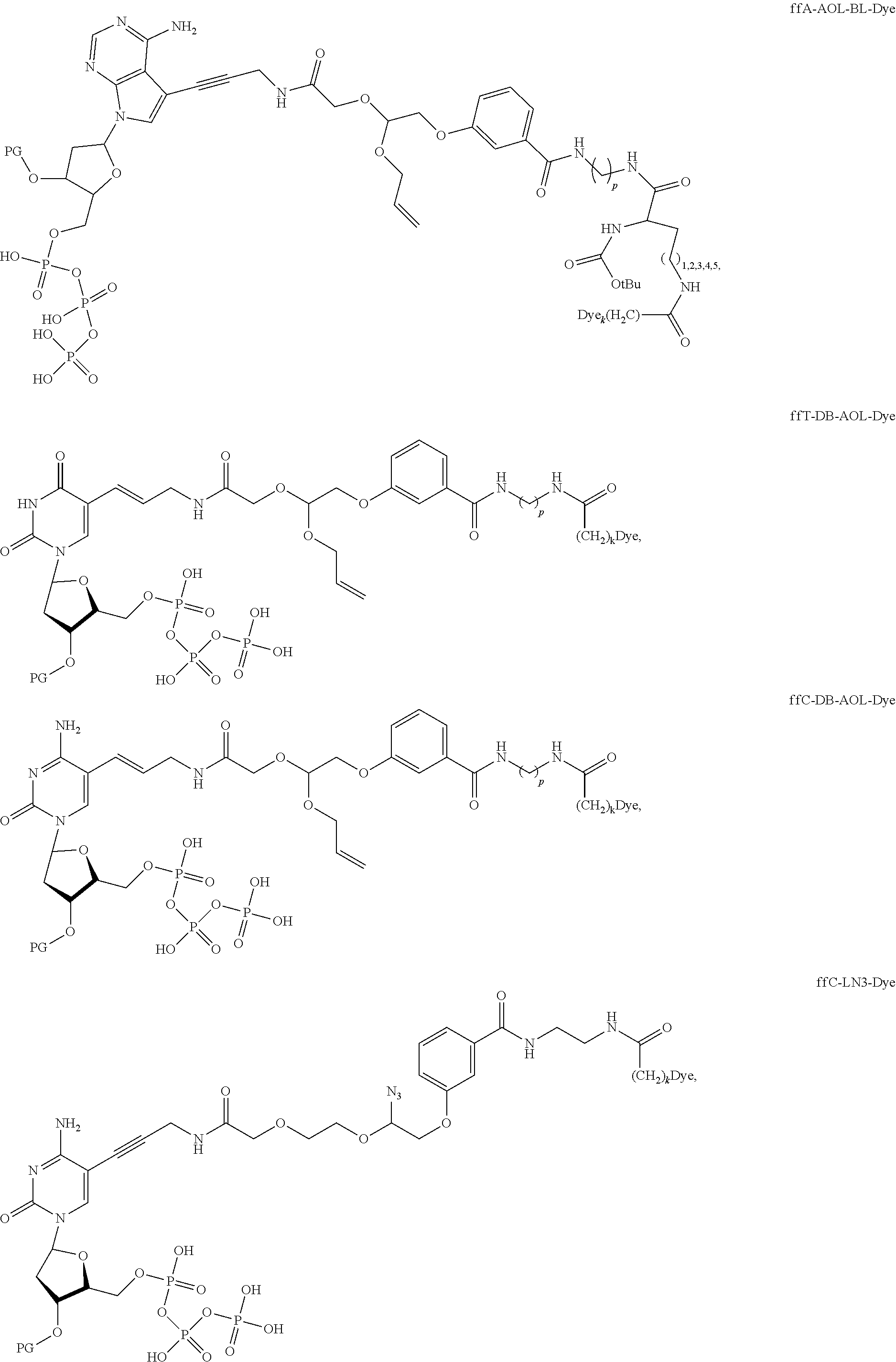

wherein PG stands for the 3′ vinyl blocking groups described herein; p is an integer of 1, 2, 3, 4, 5, 6, 7, 8, 9, or 10; and k is 0, 1, 2, 3, 4, or 5. In one embodiment, k is 5. In some further embodiments, p is 1, 2 or 3; and k is 5.

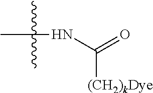

refers to the connection point of the Dye with the cleavable linker as a result of a reaction between an amino group of the linker moiety and the carboxyl group of the Dye. In any embodiments of the labeled nucleotide described herein, the nucleotide is a nucleotide triphosphate.

Various fluorescent dyes may be used in the present disclosure as detectable labels, in particularly those dyes that may be excited by a blue light (e.g., about 450 nm to about 460 nm) or a green light (e.g., about 520 nm to about 540 nm). These dyes may also be referred to as “blue dyes” and “green dyes” respectively. Examples of various type of blue dyes, including but not limited to coumarin dyes, chromenoquinoline dyes, and bisboron containing heterocycles are disclosed in U.S. Publication Nos. 2018/0094140, 2018/0201981, 2020/0277529, 2020/0277670,2021/0188832,2022/0033900,2022/0195517 A1, 2022/0380389 A1 and U.S. Ser. No. 63/325,057, each of which is incorporated by reference in its entirety. Examples of green dyes including cyanine or polymethine dyes disclosed in International Publication Nos. WO2013/041117, WO2014/135221, WO 2016/189287, WO2017/051201 and WO2018/060482A1, each of which is incorporated by reference in its entirety.

In any embodiments of nucleotide described herein, the nucleotide comprises a 2′ deoxyribose moiety (i.e., R¹ in Formula (I), (Ia) or (Id) is H, and R² is H). In some further respect, the 2′ deoxyribose contains one, two or three phosphate groups at the 5′ position of the sugar ring. In some further aspect, the nucleotides described herein are nucleotide triphosphate (i.e., —OR³ in Formula (I) and (Ia) is triphosphate).

### Biosynthesis of Oligonucleotides

Oligonucleotides are frequently used in synthetic biology. One approach to the synthesis of oligonucleotides, is the enzymatic synthesis of oligonucleotides using template-independent polymerase. Such polymerases can incorporate nucleotides to an oligonucleotide strand without need for a template strand. Template-independent polymerase may include a nucleotidyl transferase (such as terminal deoxynucleotidyl transferase (TdT)), a PolyA polymerase, and/or a CCA-adding RNA polymerase. The template-independent polymerase may be specific to RNA and/or DNA. Stable, easily removed 3′-blocks may provide a greater level of control in oligonucleotide synthesis using a template-independent polymerase.

Another aspect of the present disclosure relates to a method of controlled synthesis of an oligonucleotide or polynucleotide, comprising:
contacting a nucleotide of Formula (I), (Ia), (Ib), (Ib′) or (Ic) as described herein with the oligonucleotide or polynucleotide in the presence of a polymerase; and
incorporating the nucleotide to the 3′ end of the oligonucleotide or polynucleotide.

In some embodiments of the oligo synthesis, the method further comprises: removing the 3′vinyl blocking group of the incorporated nucleotide to generate a 3′ hydroxy group on the incorporated nucleotide, and incorporating a second nucleotide.

In some embodiments of the oligo synthesis, the polymerase is a template independent polymerase. In further embodiments, the template independent polymerase is a RNA-specific nucleotidyl transferase or a DNA-specific nucleotidyl transferase. In still further embodiments, the template independent polymerase is TdT, PolyA polymerase, or CCA-adding RNA polymerase.

In some embodiments of the oligo synthesis, the removing of the 3′vinyl blocking group of the incorporated nucleotide is achieved by a tetrazine reagent. For example, the tetrazine reagent may be selected from the group consisting of

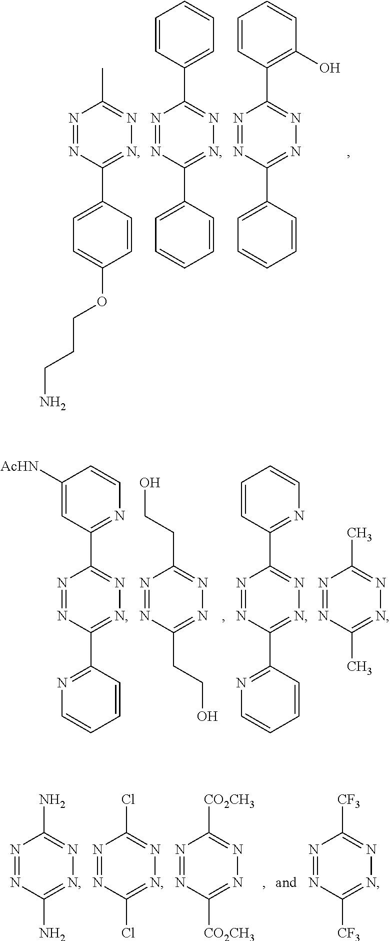

and optionally substituted variants thereof.

In some embodiments of the oligo synthesis, the incorporated nucleotide is a 2′ deoxynucleotide (i.e., R¹ is H). In other embodiments, R¹ is hydroxy, or a hydroxy protecting group, for example, a hydroxy protecting group selected from the group consisting of

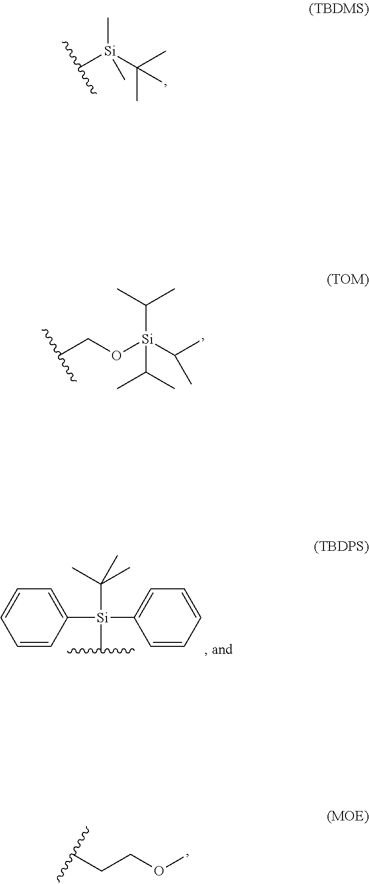

wherein the squiggly line shows the point of attachment of the protecting group to the 2′ oxygen atom. In another embodiment, R¹ is halo, such as fluoro. In still other embodiments, R¹ is —OR⁵. In some such embodiment, R⁵ is C₁-C₆ alkyl, (e.g., methyl, ethyl, n-propyl, isopropyl, n-butyl, or t-butyl). In one embodiment, R¹ is methoxy.

Synthesis of oligonucleotides may proceed via use of the 3′ vinyl blocked nucleoside triphosphates catalyzed by DNA/RNA polymerase described herein. Such a reaction with 3′ vinyl blocking group may prevent subsequent nucleoside incorporation by the polymerase, thus limiting addition to a single nucleotide. Polymerization may then only proceed after removal of the 3′ vinyl block. Once the vinyl/vinyl equivalent group is cleaved, another nucleotide may be added to the 3′ end of the oligonucleotide in the same fashion.

Scheme 1 illustrates the addition of a nucleotide to an oligonucleotide.

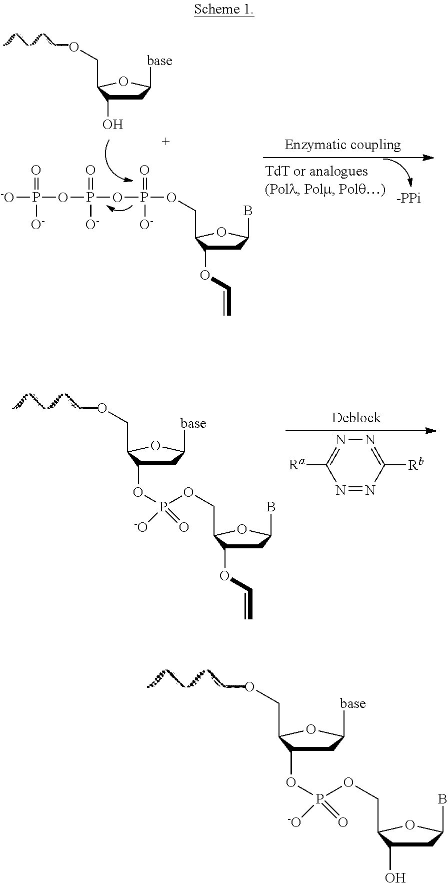

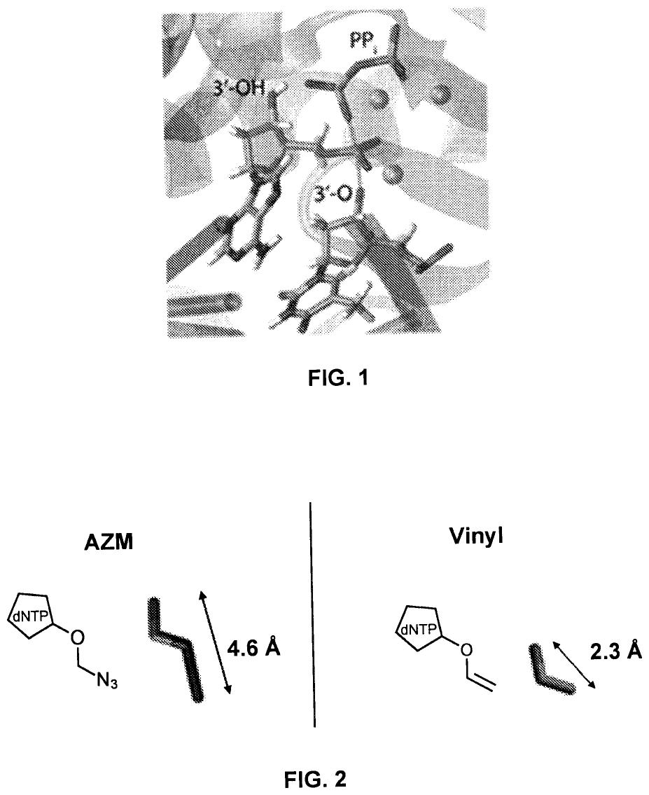

FIG. 1 illustrates a three-dimensional visualization of the reaction of a nucleoside triphosphate with a free 3′ hydroxy of growing oligonucleotide chain as catalyzed by a DNA polymerase.

Azidomethyl (AZM), another choice of blocking group, may not be suitable for certain applications. Firstly, AZM may not be stable over long time periods. Secondly, AZM is prone to incomplete removal. FIG. 2 illustrates an AZM blocking group alongside a vinyl blocking group, along with a three-dimensional visualization of the two blocking groups. The vinyl group's relatively small size may make it more compatible with enzymes capable of adding nucleotides to oligonucleotides, for example DNA polymerase or template-independent polymerases. AZM may be deblocked by THP. Though the reaction is relatively fast, THP must be present at a relatively high concentration. Additionally, THP is sensitive to O₂, which may make storage difficult. On the other hand, vinyl may be deblocked by a tetrazine reagent. The tetrazine deblocking reaction is also relatively fast, and a relatively low concentration of the tetrazine, for example from about 1 μM to about 200 μM, from about 10 μM to about 150 μM, or from about 20 to about 100 μM, may be adequate to ensure deblocking of vinyl groups, though in some cases other ranges or values may be used.

### Deprotection of the 3′ Vinyl Blocking Group

Various tetrazine species may be used to deprotect 3′ vinyl blocking group described herein. The 3′ vinyl blocking group deprotection mechanism may be similar for simple tetrazines, such as

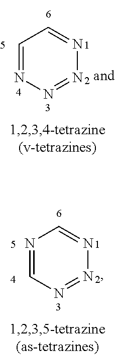 [SMILES: c1cnnnn1 ; c1ncnnn1]

and substituted tetrazines, such as

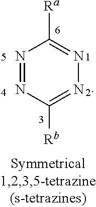

Thus, each of v-tetrazine, as-tetrazine, and/or s-tetrazine may be used in the deprotection of the 3′ vinyl blocking. Furthermore, the R^{a} and R^{b} groups of s-tetrazine may be selected to tune the reactivity and rate of the deprotection step. Generally, electron-donating groups slow down the rate of reactions but improve stability, while electron-withdrawing groups improve the rate of reactions but reduce stability. In effect, there is a trade-off between stability and reactivity of 1,2,4,5-tetrazines in an IEDDA reaction which is modulated by electron donating or electron-withdrawing groups. Generally speaking, presence of electron donating groups contributes to the stability of the compound. Presence of electron withdrawing groups reduces the stability of the compound but may increase the speed of the deprotection reaction. For example, each R^{a} and R^{b} may be independently H, C₁-C₆ alkyl, substituted C₁-C₆ alkyl, C₁-C₆ hydroxyalkyl, C₁-C₆ haloalkyl, C₁-C₆ aminoalkyl, (C₁-C₆ alkoxy)C₁-C₆ alkyl, —O—(C₁-C₆ alkoxy)C₁-C₆ alkyl, amino, substituted amino, halo, cyano, nitro, carboxyl, C-carboxy (e.g., —C(O)OC₁-C₆ alkyl), C₁-C₆alkoxy, substituted C₁-C₆ alkoxy, C₁-C₆ haloalkoxy, optionally substituted phenyl, optionally substituted 5 to 10 membered heteroaryl (e.g., pyridyl, pyrimidyl, etc.), optionally substituted C₃-C₇ cycloalkyl, or optionally substituted 3 to 10 membered heterocyclyl. In some instances, R^{a} and R^{b} are the same. In other cases, R^{a} and R^{b} are different.

### Methods of Preparation

Disclosed herein are methods of synthesizing the nucleoside/nucleotide with the 3′ vinyl blocking group. For example, a method of synthesizing a nucleotide of Formula (Id):

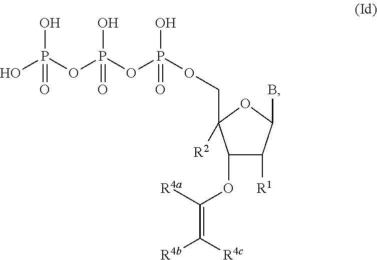

or a salt thereof, comprising:
reacting a nucleoside of Formula (Ie):

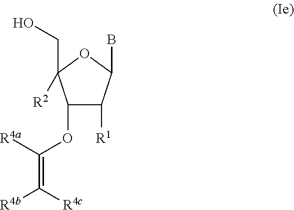

with

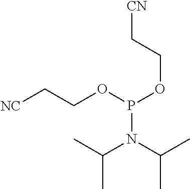 [SMILES: CC(C)N(C(C)C)P(OCCC#N)OCCC#N]

in the presence of a peroxide to form a first intermediate of Formula (Ie-1):

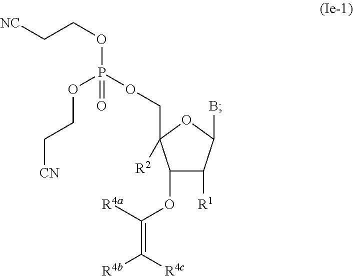

reacting the first intermediate with 1,8-diazabicyclo[5.4.0]undec-7-ene (DBU) to form a second intermediate of Formula (Ie-2):

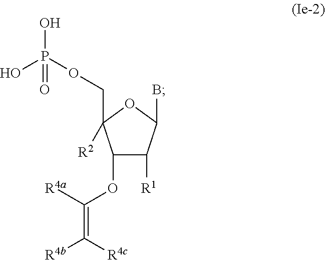

reacting the second intermediate with 2-methyl-1H-imidazole to form the third intermediate of Formula (Ie-3):

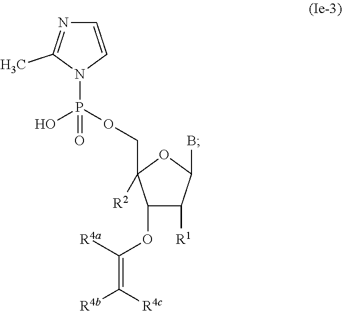

and
reacting the third intermediate with pyrophosphate H₂P₂O₇ to generate the compound of Formula (Id);
wherein B comprises a nucleobase;
R¹ is H, hydroxy, —OR⁵, halo, or a hydroxy protecting group;
R² is H;
each of R^{4a}, R^{4b} and R^{4c} is independently H, halo, C₁-C₃ alkyl, C₁-C₃ haloalkyl, C₁-C₃ hydroxyalkyl, azido, optionally substituted phenyl, optionally substituted 4 to 6 membered heteroaryl, optionally substituted C₃-C₇ cycloalkyl, or optionally substituted 3 to 7 membered heterocyclyl; and
R⁵ is C₁-C₆ alkyl or C₁-C₆ haloalkyl, or alternatively, when R¹ is —OR⁵, R⁵ and R² together with the atoms to which they are attached form a four to seven membered heterocycle containing one oxygen atom.

In some embodiments of synthetic method described herein, each of R^{4a}, R^{4b} and R^{4c} is independently H. In some embodiments, R¹ is H. In other embodiments, R¹ is hydroxy, or a hydroxy protecting group, for example, a hydroxy protecting group selected from the group consisting of

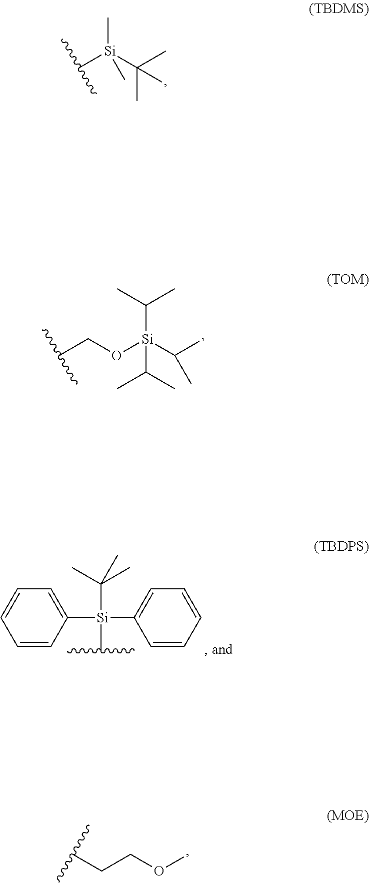

wherein the squiggly line shows the point of attachment of the protecting group to the 2′ oxygen atom. In another embodiment, R¹ is halo, such as fluoro. In still other embodiments, R¹ is —OR⁵. In some such embodiment, R⁵ is C₁-C₆ alkyl, (e.g., methyl, ethyl, n-propyl, isopropyl, n-butyl, or t-butyl). In one embodiment, R¹ is methoxy. In some embodiments, R² is H. In other embodiments, R¹ is —OR⁵ and R⁵ and R² together with the atoms to which they are attached form a four membered heterocycle

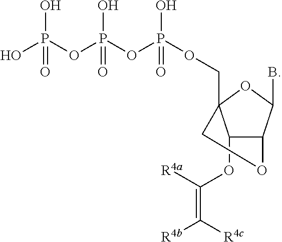

In some embodiments, the formation of intermediate Ie-3 is in the presence of an activating agent. Non-limiting examples include aldrithiol/triphenylphosphine (TPP) or trifluoroacetic anhydride (TFAA) to mix with 2NMI.

### Methods of Sequencing

Nucleotides comprising 3′ vinyl blocking group according to the present disclosure may be used in any method of analysis such as method that include detection of a fluorescent label attached to such nucleotide, whether on its own or incorporated into or associated with a larger molecular structure or conjugate. In this context the term “incorporated into a polynucleotide” can mean that the 5′ phosphate is joined in phosphodiester linkage to the 3′ hydroxyl group of a second nucleotide, which may itself form part of a longer polynucleotide chain. The 3′ end of a nucleotide set forth herein may or may not be joined in phosphodiester linkage to the 5′ phosphate of a further nucleotide. Thus, in one non-limiting embodiment, the disclosure provides a method of detecting a labeled nucleotide incorporated into a polynucleotide which comprises: (a) incorporating at least one labeled nucleotide of the disclosure into a polynucleotide and (b) determining the identity of the nucleotide(s) incorporated into the polynucleotide by detecting the fluorescent signal from the dye compound attached to said nucleotide(s).

This method can include: a synthetic step (a) in which one or more labeled nucleotides according to the disclosure are incorporated into a polynucleotide and a detection step (b) in which one or more labeled nucleotide(s) incorporated into the polynucleotide are detected by detecting or quantitatively measuring their fluorescence.

Some embodiments of the present application are directed to a method for determining the sequences of a plurality of different target polynucleotides, comprising:
(a) contacting a solid support with a solution comprising sequencing primers under hybridization conditions, wherein the solid support comprises a plurality of different target polynucleotides immobilized thereon; and the sequencing primers are complementary to at least a portion of the target polynucleotides;
(b) contacting the solid support with an aqueous solution comprising DNA polymerase and one more of four different types of nucleotides (e.g., dATP, dGTP, dCTP and dTTP or dUTP), under conditions suitable for DNA polymerase-mediated primer extension, and incorporating one type of nucleotides into the sequencing primers to produce extended copy polynucleotides, wherein at least one type of nucleotide is a labeled nucleotide (e.g., the nucleotide of Formula (I) or (Ia), in which both R¹ and R² are H; —OR³ is a triphosphate; and B comprises a nucleobase covalently attached to a detectable label), and wherein each of the four types of nucleotides comprises a 3′ vinyl blocking group;
(c) imaging the solid support and performing one or more fluorescent measurements of the extended copy polynucleotides; and
(d) removing the 3′ vinyl blocking group of the incorporated nucleotides. In some embodiments, step (d) also removes the labels of the incorporated nucleotides (if the incorporated nucleotides are labeled). In some such embodiments, the labels and the 3′ vinyl blocking groups of the incorporated nucleotides are removed in a single chemical reaction. In some further embodiments, the method may also comprises (e) washing the solid support with an aqueous wash solution (e.g., washing the removed label moiety and the 3′ blocking group away from the extended copy polynucleotides). In some embodiments, steps (b) through (e) are repeated at least 30, 40, 50, 60, 70, 80, 90, 100, 110, 120, 130, 140, 150, 160, 170, 180, 190, 200, 250, 300, 350, 400, 450, or 500 cycles to determine the target polynucleotide sequences. In some embodiments, the four types of nucleotides comprise dATP, dCTP, dGTP and dTTP or dUTP, or non-natural nucleotide analogs thereof. In some embodiments, the sequence determination is conducted after the completion of repeated cycles of the sequencing steps described herein.

In some embodiments, at least one nucleotide is incorporated into a polynucleotide (such as a single stranded primer polynucleotide described herein) in the synthetic step by the action of a polymerase enzyme. However, other methods of joining nucleotides to polynucleotides, such as, for example, chemical oligonucleotide synthesis or ligation of labeled oligonucleotides to unlabeled oligonucleotides, can be used. Therefore, the term “incorporating,” when used in reference to a nucleotide and polynucleotide, can encompass polynucleotide synthesis by chemical methods as well as enzymatic methods.

In a specific embodiment, a synthetic step is carried out and may optionally comprise incubating a template or target polynucleotide strand with a reaction mixture comprising fluorescently labeled nucleotides of the disclosure. A polymerase can also be provided under conditions which permit formation of a phosphodiester linkage between a free 3′ hydroxyl group on a polynucleotide strand annealed to the template or target polynucleotide strand and a 5′ phosphate group on the labeled nucleotide. Thus, a synthetic step can include formation of a polynucleotide strand as directed by complementary base pairing of nucleotides to a template/target strand.

In all embodiments of the methods, the detection step may be carried out while the polynucleotide strand into which the labeled nucleotides are incorporated is annealed to a template/target strand, or after a denaturation step in which the two strands are separated. Further steps, for example chemical or enzymatic reaction steps or purification steps, may be included between the synthetic step and the detection step. In particular, the polynucleotide strand incorporating the labeled nucleotide(s) may be isolated or purified and then processed further or used in a subsequent analysis. By way of example, polynucleotide strand incorporating the labeled nucleotide(s) as described herein in a synthetic step may be subsequently used as labeled probes or primers. In other embodiments, the product of the synthetic step set forth herein may be subject to further reaction steps and, if desired, the product of these subsequent steps purified or isolated.

Suitable conditions for the synthetic step will be well known to those familiar with standard molecular biology techniques. In one embodiment, a synthetic step may be analogous to a standard primer extension reaction using nucleotide precursors, including the labeled nucleotides as described herein, to form an extended polynucleotide strand (primer polynucleotide strand) complementary to the template/target strand in the presence of a suitable polymerase enzyme. In other embodiments, the synthetic step may itself form part of an amplification reaction producing a labeled double stranded amplification product comprised of annealed complementary strands derived from copying of the primer and template polynucleotide strands. Other exemplary synthetic steps include nick translation, strand displacement polymerization, random primed DNA labeling, etc. A particularly useful polymerase enzyme for a synthetic step is one that is capable of catalyzing the incorporation of the labeled nucleotides as set forth herein. A variety of naturally occurring or mutant/modified polymerases can be used. By way of example, a thermostable polymerase can be used for a synthetic reaction that is carried out using thermocycling conditions, whereas a thermostable polymerase may not be desired for isothermal primer extension reactions. Suitable thermostable polymerases which are capable of incorporating the labeled nucleotides according to the disclosure include those described in WO 2005/024010, WO06120433, and US Publication Nos. 2020/0131484 A1 and 2020/0181587 A1 each of which is incorporated herein by reference. In synthetic reactions which are carried out at lower temperatures such as 37° C., polymerase enzymes need not necessarily be thermostable polymerases, therefore the choice of polymerase will depend on a number of factors such as reaction temperature, pH, strand-displacing activity and the like.

In specific non-limiting embodiments, the disclosure encompasses methods of nucleic acid sequencing, re-sequencing, whole genome sequencing, single nucleotide polymorphism scoring, any other application involving the detection of the modified nucleotide or nucleoside labeled with dyes set forth herein when incorporated into a polynucleotide.

A particular embodiment of the disclosure provides use of labeled nucleotides comprising dye moiety according to the disclosure in a polynucleotide sequencing-by-synthesis reaction. Sequencing-by-synthesis generally involves sequential addition of one or more nucleotides or oligonucleotides to a growing polynucleotide chain in the 5′ to 3′ direction using a polymerase or ligase in order to form an extended polynucleotide chain complementary to the template/target nucleic acid to be sequenced. The identity of the base present in one or more of the added nucleotide(s) can be determined in a detection or “imaging” step. The identity of the added base may be determined after each nucleotide incorporation step. The sequence of the template may then be inferred using conventional Watson-Crick base-pairing rules. The use of the nucleotides labeled with dyes set forth herein for determination of the identity of a single base may be useful, for example, in the scoring of single nucleotide polymorphisms, and such single base extension reactions are within the scope of this disclosure.

In an embodiment of the present disclosure, the sequence of a template/target polynucleotide is determined by detecting the incorporation of one or more nucleotides into a nascent strand complementary to the template polynucleotide to be sequenced through the detection of fluorescent label(s) attached to the incorporated nucleotide(s). Sequencing of the template polynucleotide can be primed with a suitable primer (or prepared as a hairpin construct which will contain the primer as part of the hairpin), and the nascent chain is extended in a stepwise manner by addition of nucleotides to the 3′ end of the primer in a polymerase-catalyzed reaction.

In particular embodiments, each of the different nucleotide triphosphates (A, T, G and C) may be labeled with a unique fluorophore and also comprises a blocking group at the 3′ position to prevent uncontrolled polymerization. Alternatively, one of the four nucleotides may be unlabeled (dark). The polymerase enzyme incorporates a nucleotide into the nascent chain complementary to the template/target polynucleotide, and the blocking group prevents further incorporation of nucleotides. Any unincorporated nucleotides can be washed away and the fluorescent signal from each incorporated nucleotide can be “read” optically by suitable means, such as a charge-coupled device using light source excitation and suitable emission filters. The 3′ blocking group and fluorescent dye compounds can then be removed (deprotected) (simultaneously or sequentially) to expose the nascent chain for further nucleotide incorporation. Typically, the identity of the incorporated nucleotide will be determined after each incorporation step, but this is not strictly essential. Similarly, U.S. Pat. No. 5,302,509 (which is incorporated herein by reference) discloses a method to sequence polynucleotides immobilized on a solid support.

The method, as exemplified above, utilizes the incorporation of fluorescently labeled, 3′-blocked nucleotides A, G, C, and T into a growing strand complementary to the immobilized polynucleotide, in the presence of DNA polymerase. The polymerase incorporates a base complementary to the target polynucleotide but is prevented from further addition by the 3′-blocking group. The label of the incorporated nucleotide can then be determined, and the blocking group removed by chemical cleavage to allow further polymerization to occur. The nucleic acid template to be sequenced in a sequencing-by-synthesis reaction may be any polynucleotide that it is desired to sequence. The nucleic acid template for a sequencing reaction will typically comprise a double stranded region having a free 3′ hydroxyl group that serves as a primer or initiation point for the addition of further nucleotides in the sequencing reaction. The region of the template to be sequenced will overhang this free 3′ hydroxyl group on the complementary strand. The overhanging region of the template to be sequenced may be single stranded but can be double-stranded, provided that a “nick is present” on the strand complementary to the template strand to be sequenced to provide a free 3′ OH group for initiation of the sequencing reaction. In such embodiments, sequencing may proceed by strand displacement. In certain embodiments, a primer bearing the free 3′ hydroxyl group may be added as a separate component (e.g., a short oligonucleotide) that hybridizes to a single-stranded region of the template to be sequenced. Alternatively, the primer and the template strand to be sequenced may each form part of a partially self-complementary nucleic acid strand capable of forming an intra-molecular duplex, such as for example a hairpin loop structure. Hairpin polynucleotides and methods by which they may be attached to solid supports are disclosed in PCT Publication Nos. WO0157248 and WO2005/047301, each of which is incorporated herein by reference. Nucleotides can be added successively to a growing primer, resulting in synthesis of a polynucleotide chain in the 5′ to 3′ direction. The nature of the base which has been added may be determined, particularly but not necessarily after each nucleotide addition, thus providing sequence information for the nucleic acid template. Thus, a nucleotide is incorporated into a nucleic acid strand (or polynucleotide) by joining of the nucleotide to the free 3′ hydroxyl group of the nucleic acid strand via formation of a phosphodiester linkage with the 5′ phosphate group of the nucleotide.

The nucleic acid template to be sequenced may be DNA or RNA, or even a hybrid molecule comprised of deoxynucleotides and ribonucleotides. The nucleic acid template may comprise naturally occurring and/or non-naturally occurring nucleotides and natural or non-natural backbone linkages, provided that these do not prevent copying of the template in the sequencing reaction.

In certain embodiments, the nucleic acid template to be sequenced may be attached to a solid support via any suitable linkage method known in the art, for example via covalent attachment. In certain embodiments template polynucleotides may be attached directly to a solid support (e.g., a silica-based support). However, in other embodiments of the disclosure the surface of the solid support may be modified in some way so as to allow either direct covalent attachment of template polynucleotides, or to immobilize the template polynucleotides through a hydrogel or polyelectrolyte multilayer, which may itself be non-covalently attached to the solid support.

Arrays in which polynucleotides have been directly attached to a support (for example, silica-based supports such as those disclosed in WO00/06770 (incorporated herein by reference), wherein polynucleotides are immobilized on a glass support by reaction between a pendant epoxide group on the glass with an internal amino group on the polynucleotide. In addition, polynucleotides can be attached to a solid support by reaction of a sulfur-based nucleophile with the solid support, for example, as described in WO2005/047301 (incorporated herein by reference). A still further example of solid-supported template polynucleotides is where the template polynucleotides are attached to hydrogel supported upon silica-based or other solid supports, for example, as described in WO00/31148, WO01/01143, WO02/12566, WO03/014392, U.S. Pat. No. 6,465,178 and WO00/53812, each of which is incorporated herein by reference.

A particular surface to which template polynucleotides may be immobilized is a polyacrylamide hydrogel. Polyacrylamide hydrogels are described in the references cited above and in WO2005/065814, which is incorporated herein by reference. Specific hydrogels that may be used include those described in WO2005/065814 and U.S. Pub. No. 2014/0079923. In one embodiment, the hydrogel is PAZAM (poly(N-(5-azidoacetamidylpentyl) acrylamide-co-acrylamide)).

DNA template molecules can be attached to beads or microparticles, for example, as described in U.S. Pat. No. 6,172,218 (which is incorporated herein by reference). Attachment to beads or microparticles can be useful for sequencing applications. Bead libraries can be prepared where each bead contains different DNA sequences. Exemplary libraries and methods for their creation are described in Nature, 437, 376-380 (2005); Science, 309, 5741, 1728-1732 (2005), each of which is incorporated herein by reference. Sequencing of arrays of such beads using nucleotides set forth herein is within the scope of the disclosure.

Template(s) that are to be sequenced may form part of an “array” on a solid support, in which case the array may take any convenient form. Thus, the method of the disclosure is applicable to all types of high-density arrays, including single-molecule arrays, clustered arrays, and bead arrays. Nucleotides labeled with dye compounds of the present disclosure may be used for sequencing templates on essentially any type of array, including but not limited to those formed by immobilization of nucleic acid molecules on a solid support.

However, nucleotides labeled with dye compounds of the disclosure are particularly advantageous in the context of sequencing of clustered arrays. In clustered arrays, distinct regions on the array (often referred to as sites, or features) comprise multiple polynucleotide template molecules. Generally, the multiple polynucleotide molecules are not individually resolvable by optical means and are instead detected as an ensemble. Depending on how the array is formed, each site on the array may comprise multiple copies of one individual polynucleotide molecule (e.g., the site is homogenous for a particular single- or double-stranded nucleic acid species) or even multiple copies of a small number of different polynucleotide molecules (e.g., multiple copies of two different nucleic acid species). Clustered arrays of nucleic acid molecules may be produced using techniques generally known in the art. By way of example, WO 98/44151 and WO00/18957, each of which is incorporated herein, describe methods of amplification of nucleic acids wherein both the template and amplification products remain immobilized on a solid support in order to form arrays comprised of clusters or “colonies” of immobilized nucleic acid molecules. The nucleic acid molecules present on the clustered arrays prepared according to these methods are suitable templates for sequencing using nucleotides labeled with dye compounds of the disclosure.

Nucleotides labeled with dye compounds of the present disclosure are also useful in sequencing of templates on single molecule arrays. The term “single molecule array” or “SMA” as used herein refers to a population of polynucleotide molecules, distributed (or arrayed) over a solid support, wherein the spacing of any individual polynucleotide from all others of the population is such that it is possible to individually resolve the individual polynucleotide molecules. The target nucleic acid molecules immobilized onto the surface of the solid support can thus be capable of being resolved by optical means in some embodiments. This means that one or more distinct signals, each representing one polynucleotide, will occur within the resolvable area of the particular imaging device used.

Single molecule detection may be achieved wherein the spacing between adjacent polynucleotide molecules on an array is at least 100 nm, more particularly at least 250 nm, still more particularly at least 300 nm, even more particularly at least 350 nm. Thus, each molecule is individually resolvable and detectable as a single molecule fluorescent point, and fluorescence from said single molecule fluorescent point also exhibits single step photobleaching.

The terms “individually resolved” and “individual resolution” are used herein to specify that, when visualized, it is possible to distinguish one molecule on the array from its neighboring molecules. Separation between individual molecules on the array will be determined, in part, by the particular technique used to resolve the individual molecules. The general features of single molecule arrays will be understood by reference to published applications WO00/06770 and WO 01/57248, each of which is incorporated herein by reference. Although one use of the labeled nucleotides of the disclosure is in sequencing-by-synthesis reactions, the utility of such nucleotides is not limited to such methods. In fact, the labeled nucleotides described herein may be used advantageously in any sequencing methodology which requires detection of fluorescent labels attached to nucleotides incorporated into a polynucleotide.

Alternatively, the sequencing methods described herein may also be carried out using unlabeled nucleotides and affinity reagents containing a fluorescent dye described herein. For example, one, two, three or each of the four different types of nucleotides (e.g., dATP, dCTP, dGTP and dTTP or dUTP) in the incorporation mixture of step (a) may be unlabeled. Each of the four types of nucleotides (e.g., dNTPs) has a 3′ vinyl blocking group to ensure that only a single base can be added by a polymerase to the 3′ end of the primer polynucleotide. After incorporation of an unlabeled nucleotide in step (b), the remaining unincorporated nucleotides are washed away. An affinity reagent is then introduced that specifically recognizes and binds to the incorporated dNTP to provide a labeled extension product comprising the incorporated dNTP. Uses of unlabeled nucleotides and affinity reagents in sequencing by synthesis have been disclosed in WO 2018/129214 and WO 2020/097607. A modified sequencing method of the present disclosure using unlabeled nucleotides may include the following steps:
(a′) contacting a solid support with a solution comprising sequencing primers under hybridization conditions, wherein the solid support comprises a plurality of different target polynucleotides immobilized thereon; and the sequencing primers are complementary to at least a portion of the target polynucleotides;
(b′) contacting the solid support with an aqueous solution comprising DNA polymerase and one more of four different types of unlabeled nucleotides (e.g., dATP, dCTP, dGTP, and dTTP or dUTP) under conditions suitable for DNA polymerase-mediated primer extension, and incorporating one type of nucleotides into the sequencing primers to produce extended copy polynucleotides, and wherein each of the four types of nucleotides comprises a 3′ vinyl blocking group;
(c′) contacting the extended copy polynucleotides with a set of affinity reagents under conditions wherein one affinity reagent binds specifically to the incorporated unlabeled nucleotides to provide labeled extended copy polynucleotides;
(d′) imaging the solid support and performing one or more fluorescent measurements of the extended copy polynucleotides; and
(e′) removing the 3′ vinyl blocking group of the incorporated nucleotides.

In some embodiments of the modified sequencing method described herein, the method further comprises removing the affinity reagents from the incorporated nucleotides. In still further embodiments, the 3′ vinyl blocking group and the affinity reagent are removed in the same reaction. In some embodiments, the method further comprises a step (f′) washing the solid support with an aqueous wash solution. In further embodiments, steps (b′) through (f′) are repeated at least 50, 100, 150, 200, 250, 300, 350, 400, 450, or 500 cycles to determine the target polynucleotide sequences. In some embodiments, the set of affinity reagents may comprise a first affinity reagent that binds specifically to the first type of nucleotide, a second affinity reagent that binds specifically to the second type of nucleotide, and a third affinity reagent that binds specifically to the third type of nucleotide. In some further embodiments, each of the first, second and the third affinity reagents comprises a detectable labeled that is spectrally distinguishable. In some embodiments, the affinity reagents may include protein tags, antibodies (including but not limited to binding fragments of antibodies, single chain antibodies, bispecific antibodies, and the like), aptamers, knottins, affimers, or any other known agent that binds an incorporated nucleotide with a suitable specificity and affinity. In one embodiment, at least one affinity reagent is an antibody or a protein tag.

### EXAMPLES

### Example 1. Synthesis of 3′Vinyl Blocked dTTP

A 3′ vinyl protected T nucleoside (Compound 3) was synthesized by two different synthetic routes illustrated in Schemes 3 and Scheme 4 respectively.

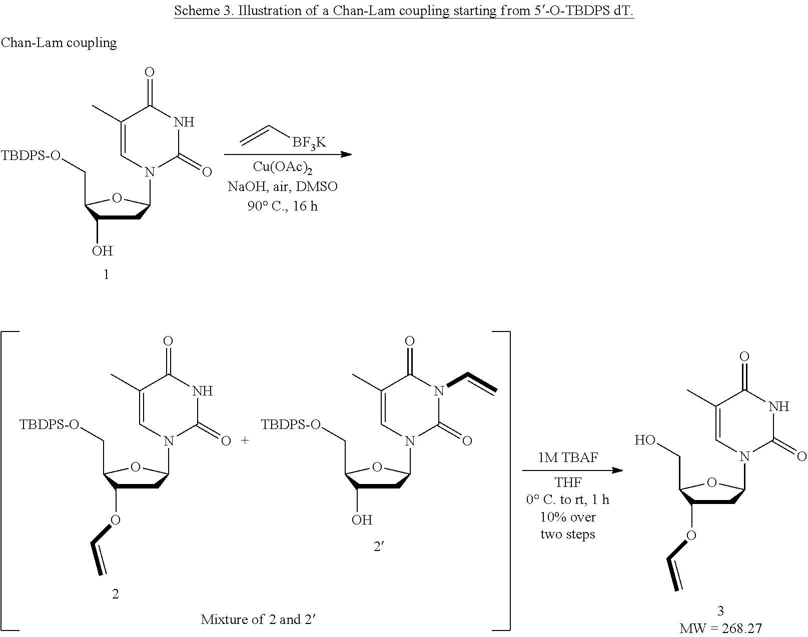

To a solution of 5′-O-TBDPS-dT nucleoside 1 (0.5 mmol, 480.64 g/mol) in DMSO containing molecular sieves (10.0 mL), under air atmosphere and at room temperature were added potassium vinyl trifluoroborate (1 mmol, 133.95 g/mol), copper acetate (10 mol %, 123.60 g/mol) and powdered NaOH (1.0 mmol, 40 g/mol). The reaction mixture was heated gently to 90° C. for 16 h at which point TLC showed the complete conversion of alcohol 1 to the vinylated products 2 and 2′. After cooling to room temperature, ice cold water was added dropwise to the reaction mixture, then filtered and diluted with EtOAc. The organic layer was washed twice with water and brine before being dried over anhydrous sodium sulphate. The drying agent was removed by filtration, and the solvent was evaporated under vacuum to give a crude colorless oil which was directly dissolved in THF, cooled to 0° C. and treated with TBAF (1 mmol, 1M). The reaction mixture was then warmed to room temperature and stirred for additional 1 h, quenched with ice cold water and diluted with EtOAC. The organic layer was washed twice with water and brine before being dried over anhydrous sodium sulphate. The drying agent was removed by filtration and the solvent was evaporated under vacuum to give a crude colorless oil purified by silica gel chromatography (hexane/EtOAc=2:3) to yield 3′ vinyl blocked compound 3 as a pale-yellow oil (10% over two steps). The purified fractions were characterized by ¹H NMR and MS to confirm the structure 3. ESI-MS (−ve mode) m/z 267.1 [M-H]—.

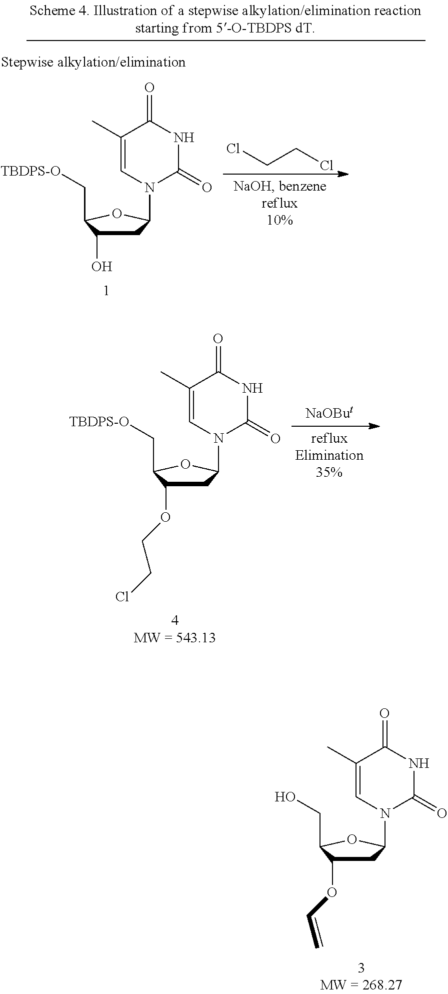

To a solution of 5′-O-TBDPS-dT nucleoside 1 (5.41 mmol, 480.64 g/mol) in benzene (20 mL) and 1,2-dichloroethane (10.0 mL) was added powdered NaOH (10.82 mmol, 40 g/mol) under argon atmosphere at room temperature. The reaction mixture was gently refluxed to 90° C. for 16 h to 76 h, at which point TLC showed ˜15% conversion of compound 1 to the 3′-alkylated product 4. After cooling to room temperature, ice cold water was added dropwise to the reaction mixture, then filtered and diluted with EtOAc. The organic layer was washed twice with water and brine before being dried over anhydrous sodium sulphate. The drying agent was removed by filtration and the solvent was evaporated under vacuum to give a crude colorless oil, purified by silica gel chromatography (hexane/EtOAc=1:4) to yield alkylated compound 4 as pale-yellow oil (10%). The purified fractions were characterized by ¹H NMR and MS to confirm the structure 4. ESI-MS (−ve mode) m/z 542.2 [M-H]—.

To ether 4 (0.184 mmol, 542.2 g/mol) dissolved in THF (3.0 mL) was added sodium tert-butoxide (1 mmol, 96.10 g/mol). The reaction mixture was gently refluxed for 3 h at which point TLC showed the complete conversion to the 3′vinyl blocked compound 3. After cooling to room temperature, ice cold water was added dropwise to the reaction mixture, then filtered and diluted with EtOAc. The organic layer was washed twice with water and brine before being dried over anhydrous sodium sulphate. The drying agent was removed by filtration and the solvent was evaporated under vacuum to give a crude colorless oil purified by silica gel chromatography (hexane/EtOAc=2:3) to yield 3′-vinyl compound 3 as a pale-yellow oil (35%). The purified fractions were characterized by ¹H NMR and MS to confirm the structure of compound 3.

Compound 3 was subsequently used as a starting point to synthesize 3′vinyl deoxythymidine triphosphate (dTTP), which was illustrated in Scheme 5.

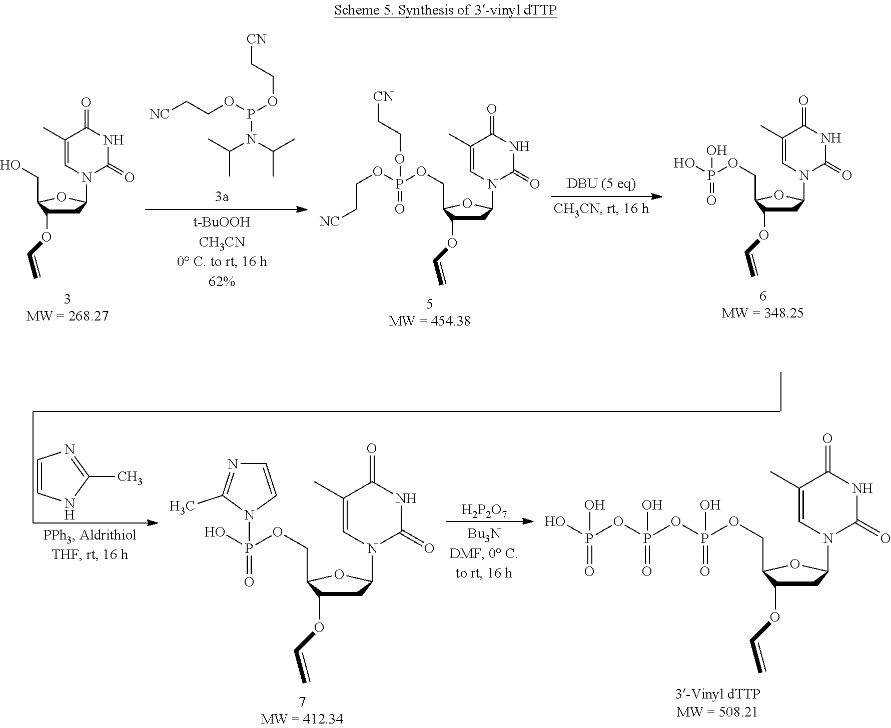

Alcohol 3 (75 μmol, 268 g/mol) and 5-(ethylthio)-1H-tetrazole (110 μmol, 130.17 g/mol) were co-evaporated with dry acetonitrile (3×1 mL) before being dissolved in CH₂Cl₂/CH₃CN (1:1, 4 mL). Under an atmosphere of dry argon, a freshly prepared reaction mixture of phosphoramidite 3a (110 μmol, 271.3 g/mol.) in acetonitrile (2 mL) was added to the above reaction mixture and stirred overnight at room temperature at which point TLC showed the complete conversion of alcohol 3 to phosphorylated compound 5. Upon cooling to 0° C., t-BuOOH (110 μmol, 6 M) was added and the mixture was stirred for 5 min at 0° C. until TLC confirmed complete oxidation. The solvent was removed under vacuum to give a crude colorless oil which was purified by silica gel chromatography (hexane/EtOAc=1:4) to yield phosphorylated product 5 as an off-white foam (62%).

To phosphorylated compound 5 (110 μmol, 454.38 g/mol) dissolved in CH₃CN (200 μL) under an atmosphere of dry argon was added DBU (550 μmol, 0.1 M in acetonitrile) and the reaction mixture was stirred overnight at room temperature at which point TLC showed the complete conversion to monophosphate 6. The solvent was removed under the vacuum to give a crude colorless oil which was purified by RP-HPLC. The purified fractions were characterized by MS and lyophilized to obtain monophosphate 6.

Monophosphate 6 (100 μmol, 348.25 g/mol) was co-evaporated with dry acetonitrile (3×1 mL) before being dissolved in DMF (500 μL). Under an atmosphere of dry argon were added 2-methyl imidazole (300 μmol, 83.2 g/mol), triphenyl phosphine (300 μmol, 262 g/mol) followed by aldrithiol (300 μmol, 220.1 g/mol) and triethylamine (500 μmol, 101.2 g/mol). The solution turned to yellow and the reaction mixture was stirred overnight at room temperature at which point MS showed the complete conversion of monophosphate 6 to the activated intermediate 7. Upon cooling to 0° C., pyrophosphate (500 μmol, 528.3 g/mol) and an excess of tributylamine were added and the mixture was stirred at 0° C. until MS confirmed the formation of the corresponding triphosphate (˜16 h). The solvent was removed under vacuum to give a crude colorless oil which was purified by RP-HPLC and the isolated fractions were characterized by MS and HPLC to confirm the formation of 3′vinyl dTTP. ESI-MS (−ve mode) m/z 507.27 [M-H]—.

### Example 2. Incorporation of 3′ Vinyl dTTP by DNA Polymerase

In this incorporation experiment, several DNA polymerases were used to test the feasibility of incorporating a 3′vinyl dTTP into a primer strand through template-dependent synthesis. For each test, 3′vinyl dTTP or an unblocked dTTP was exposed to one polymerase in the presence of a template strand having five A nucleotides overhang at the 5′ end, and the complementary primer strand has a fluorescent tag (FAM) at the 5′ end for visualization purposes. The DNA polymerases tested were Pol 812, Pol 963, Pol 1558, and Pol 1901, each at 0.12 mg/mL. The amino acid sequences of these mutants of 9° N polymerases are disclosed, for example, in U.S. Patent Publication Nos. 2020/0131484 A1 and 2020/0181587 A1, both of which are incorporated by reference herein in their entireties. The nucleotides include a 3′vinyl dTTP

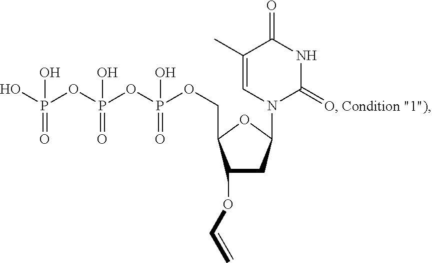

a negative control nucleotide

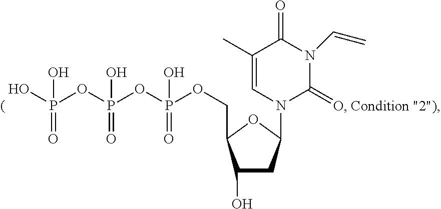

and an unaltered dTTP

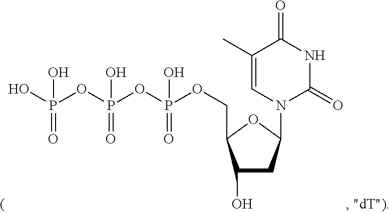 [SMILES: Cc1cn([C@H]2C[C@@H](O)[C@@H](COP(=O)(O)OP(=O)(O)OP(=O)(O)O)O2)c(=O)[nH]c1=O]

The negative control nucleotide included a vinyl blocking group attached to the nitrogen atom of the T base typically involved in base-pair binding. Thus, it was expected that the negative control nucleotide would not be able to bind with the available adenosine bases of the template strand. The unaltered dTTP, on the other hand, was expected to be incorporated into the primer strand and could therefore act as a control for 5 incorporations.

For each test reaction, Mg²⁺ concentration was at approximately 4 mM, while the polymerase of choice was at a concentration of 0.12 mg/mL. The reactions also had 20 μmol of nucleotide, nucleotide 1 or nucleotide 2 as diagrammed, and 4 μmol of the template strand (SEQ ID No. 1) and 4 μmol of the primer strand (SEQ ID No. 2). The reaction was carried at 60° C. for 10 minutes.

SEQ ID No. 1: 3′ TAAGTCCTGCTCGGAGTCTGGGAAAAA
SEQ ID No. 2: 5′ FAM - ATTCAGGACGAGCCTCAGACCC

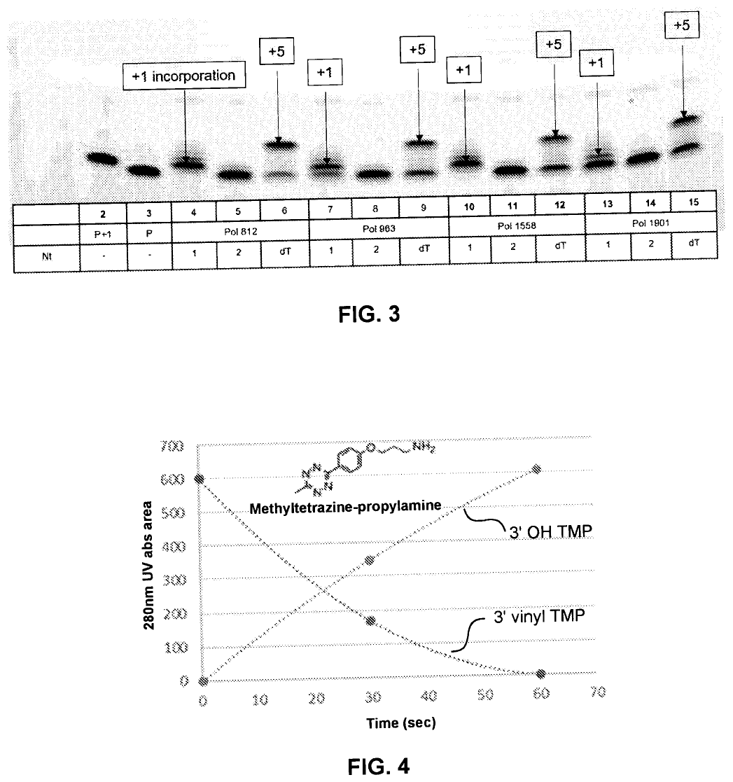

The resulting nucleic acids were run through a gel and imaged as shown in FIG. 3. Lane 2 shows the positive control when one T is added to the primer strand, while Lane 3 shows the primer strand without any additional nucleotides. Lanes 4, 7, 10, and 13 show the nucleic acids formed when nucleotide 1 was available. Lanes 5, 8, 11, and 14 show the nucleic acids formed when nucleotide 2 was available. Lanes 6, 9, 12, and 16 show the nucleic acids formed when unaltered dTTP was available.

As expected, the polymerases were not generally able to incorporate nucleotide 2 into the primer strand. On the other hand, the polymerases were able to incorporate up to a maximum of 5 unaltered dTTP into the primer strand. Each polymerase showed different capability with respect to incorporating nucleotide 1 into the primer strand. Pol 1901 was the least capable of the four, with Pol 812 and Pol 1558 appeared to have the best performance.

### Example 3. Deblocking of 3′ Vinyl Group

In this example, a preliminary deblocking test was conducted and the results of were shown in FIG. 4. 50 μM of 3′ vinyl thymidine monophosphate (TMP) was deblocked in the presence of 5 mM of methyltetrazine propylamine HCl. FIG. 4 shows that the proportion of 3′OH TMP, as measured by UV absorption at 280 nm wavelength on multiple HPLC runs, increases over time (from 0 to 60 Secs) when exposed to 5 mM of methyltetrazine propylamine HCl, until a maximum when all of the 3′vinyl TMP have been consumed or deblocked.

### Example 4. Template-Independent Enzymatic Synthesis of Oligonucleotides

A preliminary test of template-independent synthesis of oligonucleotides using a commercially available TdT was conducted. A primer strand with a FAM fluorescent tag at the 5′ end (SED ID No. 2) was introduced.

SEQ ID No. 2: 5′ FAM - ATTCAGGACGAGCCTCAGACCC

Three different nucleotides were included, depending on the test condition: a 3′ vinyl blocked dTTP (“3VT”), a 3′-vinyl blocked dTTP that is pre-heated at 60° C. for 5 minutes (“HVT”), and an unaltered dTTP with 3′ hydroxy (“dT”). HVT condition was used to test the thermal stability of the nucleotide. Each nucleotide was allowed to react with the template in the presence of the TdT for 5 or 10 minutes at room temperature. It was expected that the dT condition would result in relatively long nucleic acids, given that the unaltered dTTPs did not include any 3′blocking group.

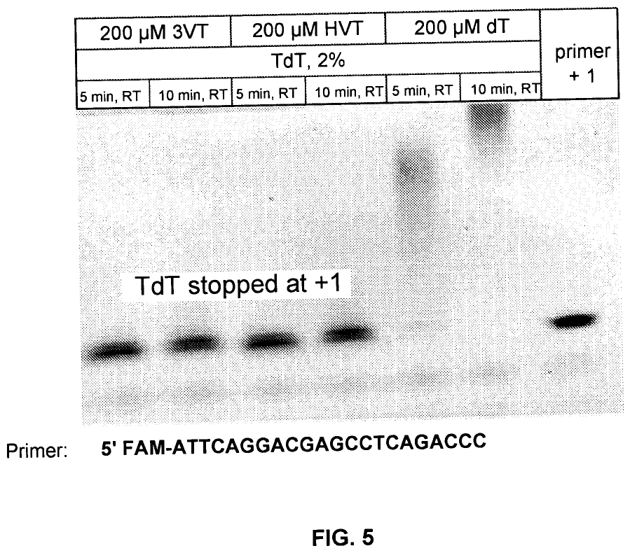

The resulting nucleic acids were run through a gel and subsequently imaged as shown in FIG. 5. A control was run simultaneously, which included a primer with one added nucleotide. At either run time, the 3VT and HVT conditions resulted in addition of just one additional nucleotide. Without being bound to a particular theory, it is believed that the 3′ blocking group on the 3VT nucleotide prevented synthesis beyond the addition of one nucleotide. In contrast, the dT condition resulted in much longer nucleic acids. These test results indicate that the commercial TdT was able to incorporate 3′blocked nucleotides despite the presence of the blocking group.

1. A nucleoside or nucleotide having the structure of Formula (I): 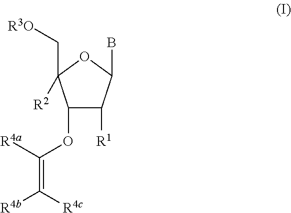
or a salt thereof, wherein
B comprises a nucleobase;
R¹ is H, hydroxy, —OR⁵, halo, or a hydroxy protecting group;
R² is H;
R³ is H, a hydroxy protecting group, or —OR³ is a monophosphate, diphosphate, triphosphate or phosphorothioate;
each of R^{4a}, R^{4b} and R^{4c} is independently H, halo, C₁-C₃ alkyl, C₁-C₃ haloalkyl, C₁-C₃ hydroxyalkyl, azido, optionally substituted phenyl, optionally substituted 5 to 6 membered heteroaryl, optionally substituted C₃-C₇ cycloalkyl, or optionally substituted 3 to 7 membered heterocyclyl; and
R⁵ is C₁-C₆ alkyl or C₁-C₆ haloalkyl, or when R¹ is —OR⁵, R⁵ and R² together with the atoms to which they are attached form a four to seven membered heterocycle containing one oxygen atom.

2. The nucleoside or nucleotide of claim 1, wherein each of R^{4a}, R^{4b} and R^{4c} is H, and the nucleoside or nucleotide has the structure of Formula (Ia): 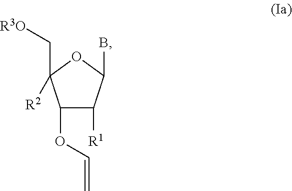
or a salt thereof.

3. The nucleoside or nucleotide of claim 1, wherein at least one of R^{4a}, R^{4b} and R^{4c} is methyl, ethyl, n-propyl, isopropyl, fluoro, chloro, —CHF₂, —CH₂F, —CH₂C₁, —CHCl₂, or —CF₃.

4. The nucleoside or nucleotide of claim 1, wherein R¹ is H.

5. The nucleoside or nucleotide of claim 1, wherein R¹ is hydroxy, or a hydroxy protecting group selected from the group consisting of 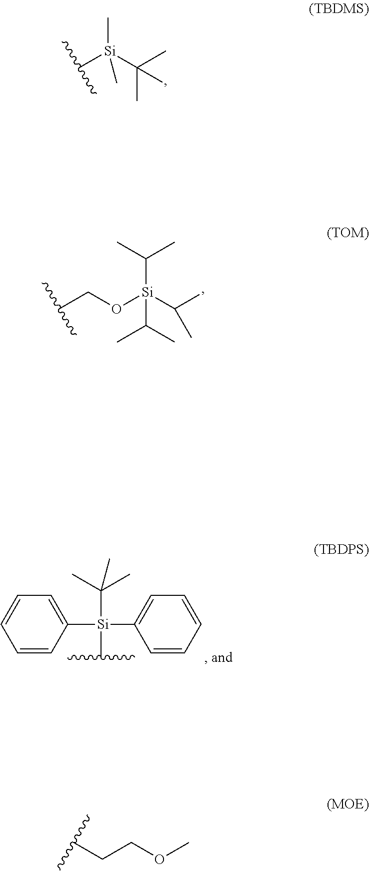
covalently attached to the 2′ oxygen atom.

6. The nucleoside or nucleotide of claim 1, wherein R¹ is fluoro.

7. The nucleoside or nucleotide of claim 1, wherein R¹ is —OR⁵ and R⁵ is methyl.

8. The nucleoside or nucleotide of claim 1, wherein R² is H.

9. The nucleoside or nucleotide of claim 1, wherein R¹ is —OR⁵ and R⁵ and R² together with the atoms to which they are attached form a four to seven membered heterocycle having the Formula (Ib): 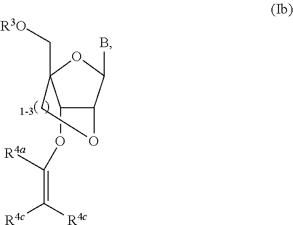
or a salt thereof.

10. The nucleoside or nucleotide of claim 9, having the structure of Formula (Ib′): 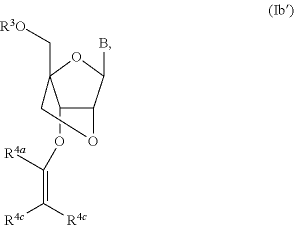
or a salt thereof.

11. The nucleoside or nucleotide of claim 10, wherein each of R^{4a}, R^{4b} and R^{4c} is H, and the nucleoside or nucleotide has the structure of Formula (Ic): 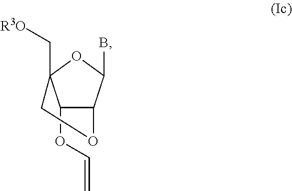
or a salt thereof.

12. The nucleoside or nucleotide of claim 1, wherein —OR³ is a monophosphate, diphosphate or triphosphate.

13. The nucleoside of claim 1, wherein the nucleobase is: 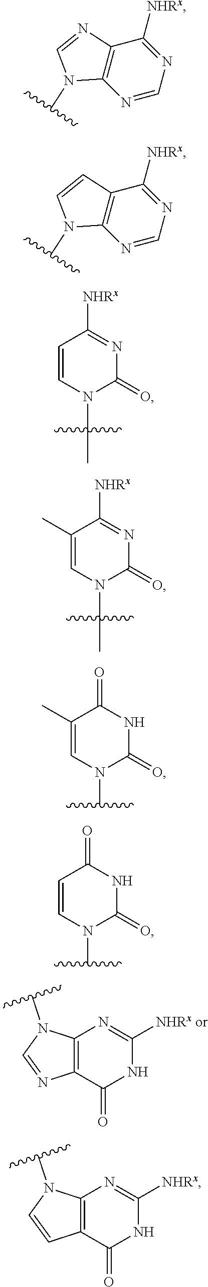
wherein R^{x} is H or an amino protecting group, or the hydrogen in —NHR^{x} is absent and R^{x} is a divalent amino protecting group.

14. The nucleoside or nucleotide of claim 13, wherein R^{x} is H, —C(═O)C₁₋₆ alkyl or —C(═O)-phenyl.

15. The nucleoside or nucleotide of claim 1, wherein B comprises a nucleobase covalently bounded to a detectable label, optionally through a linker.

16. The nucleoside or nucleotide of claim 15, B is

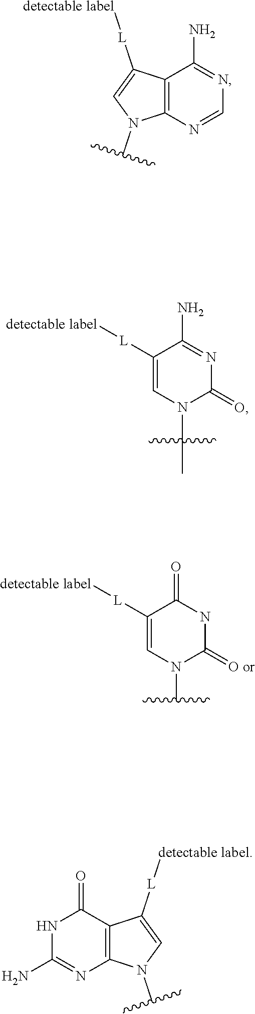

17. The nucleoside or nucleotide of claim 1, wherein the nucleoside or nucleotide is nucleotide, and —OR³ is triphosphate.

18. A method of controlled synthesis of an oligonucleotide or polynucleotide, comprising:
contacting a nucleotide of claim 17 with the oligonucleotide or polynucleotide in the presence of a polymerase; and
incorporating the nucleotide to the 3′ end of the oligonucleotide or polynucleotide.

19. The method of claim 18, further comprising:
removing the 3′vinyl blocking group of the incorporated nucleotide to generate a 3′ hydroxy group on the incorporated nucleotide, and
incorporating a second nucleotide.

20. The method of claim 18, wherein the polymerase is a template independent polymerase.

21. (canceled)

22. The method of claim 20, wherein the template independent polymerase is terminal deoxynucleotidyl transferase (TdT), PolyA polymerase, or CCA-adding RNA polymerase.

23. The method of claim 19, wherein the removing of the 3′vinyl blocking group of the incorporated nucleotide is achieved by a tetrazine reagent.

24. The method of claim 23, wherein the tetrazine reagent is selected from the group consisting of 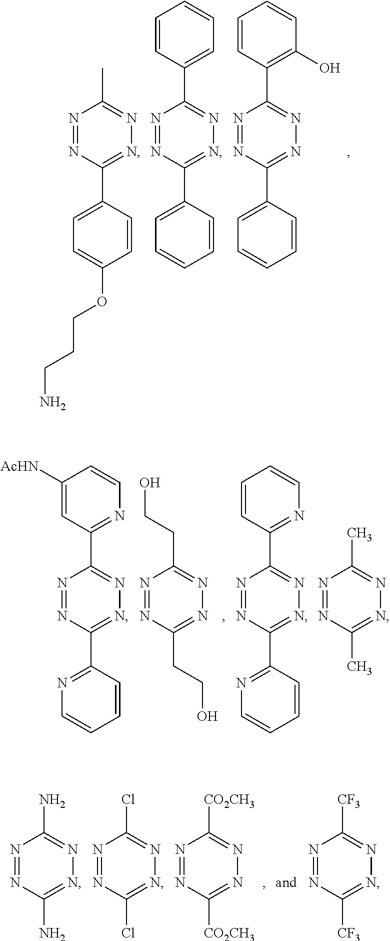
and optionally substituted variants thereof.

25. (canceled)

26. A method of synthesizing a nucleotide of Formula (Id): 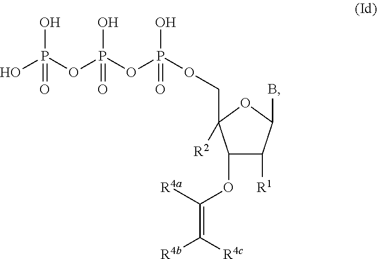
or a salt thereof, comprising:
reacting a nucleoside of Formula (Ie): 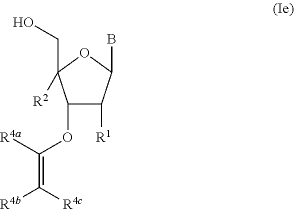
with 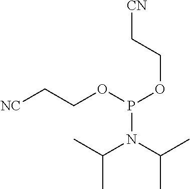 [SMILES: CC(C)N(C(C)C)P(OCCC#N)OCCC#N]
in the presence of a peroxide to form a first intermediate of Formula (Ie-1): 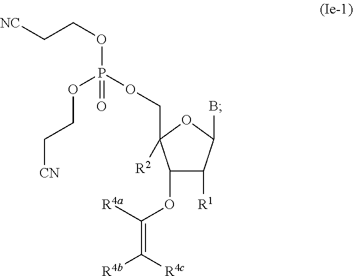
reacting the first intermediate with 1,8-diazabicyclo[5.4.0]undec-7-ene (DBU) to form a second intermediate of Formula (Ie-2): 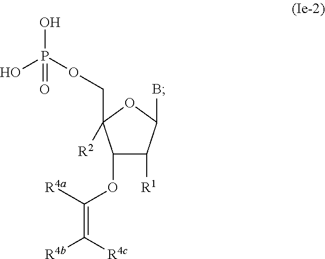
reacting the second intermediate with 2-methyl-1H-imidazole to form the third intermediate of Formula (Ie-3): 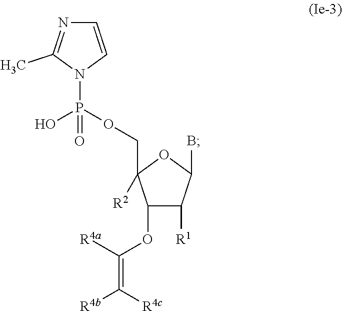
and
reacting the third intermediate with pyrophosphate H₂P₂O₇ to generate the compound of Formula (Id);
wherein B comprises a nucleobase;
R¹ is H, hydroxy, —OR⁵, halo, or a hydroxy protecting group;
R² is H;
each of R^{4a}, R^{4b} and R^{4c}, is independently H, halo, C₁-C₃ alkyl, C₁-C₃ haloalkyl, C₁-C₃ hydroxyalkyl, azido, optionally substituted phenyl, optionally substituted 4 to 6 membered heteroaryl, optionally substituted C₃-C₇ cycloalkyl, or optionally substituted 3 to 7 membered heterocyclyl; and
R⁵ is C₁-C₆ alkyl or C₁-C₆ haloalkyl, or alternatively, when R¹ is —OR⁵, R⁵ and R² together with the atoms to which they are attached form a four to seven membered heterocycle containing one oxygen atom.

27. (canceled)

28. (canceled)


SEQUENCE LISTING (2 sequences)

SEQ ID NO: 1 - DNA, 27 bp, synthetic construct
  source 1..27: mol_type = other DNA
taagtcctgctcggagtctgggaaaaa

SEQ ID NO: 2 - DNA, 22 bp, synthetic construct
  misc_feature 1: note = fluorophore
  source 1..22: mol_type = other DNA
attcaggacgagcctcagaccc